(19) United States
(12) Patent Application Publication
(10) Pub. No.: US 2025/0198902 A1
(43) Pub. Date: Jun. 19, 2025

(54) METHOD OF ANALYSING THE MOTIONAL ACTIVITY OF PARTICLES

(71) Applicant: RESISTELL AG, Muttenz (CH)

(72) Inventors: Grzegorz JOZWIAK, Muttenz (CH); Alexander STURM, Muttenz (CH); Eric DELARZE, Muttenz (CH); Gino Sebastian CATHOMEN, Muttenz (CH); Danuta CICHOCKA, Muttenz (CH)

(73) Assignee: RESISTELL AG, Muttenz (CH)

(21) Appl. No.: 18/845,971

(22) PCT Filed: Mar. 6, 2023
(86) PCT No.: PCT/EP2023/055596

§ 371 Date: Sep. 11, 2024

(30) Foreign Application Priority Data
Mar. 15, 2022 (EP) 22162147.7

(51) Int. Cl. G01N 15/10; G01N 33/487

(52) U.S. Cl. CPC G01N 15/10; G01N 33/487; G01N 2015/1027

(57) ABSTRACT

A method of analysing a motional activity of particles with a motion detector including the steps of bringing at least one particle into contact with a flexible support, detecting at least one time-dependent signal that is indicative of deflections of the flexible support due to motions of the at least one particle, and evaluating the detected time-dependent signal. The evaluation includes analysing a time-dependency of the signal to derive a plurality of signal parameters from the signal that characterise a variation of the signal as a function of time, and executing a linking algorithm that has, as input variables, an input vector having at least a selection of the signal parameters, and has, as an output variable, at least one activity indicator that is indicative of a motional activity of the particle.

### TECHNICAL FIELD

The present invention relates to a method of analysing a motional activity of particles with a motion detector. In particular, the present invention relates to the analysis of the motional activity of particles exhibiting a motion at a very low scale, for instance having a size ranging from Angstroms to micrometres. It particularly but not exclusively relates to the analysis of the movement or an inner dynamics of said particles.

### PRIOR ART

Nano-mechanical oscillators have been widely used as sensors to detect small variations of a mass of a particle. In WO 2013/054311, microcantilevers are used to detect and measure the nano-scale vibrations (or nanomotions) of particles attached to their surface. Said particles can be of chemical or biological nature, including proteins, lipids, glucides, viruses, bacteria, yeasts, or mammalian cells among others. This system allows discriminating between states of the tested particles in presence or absence of stimuli allowing, for instance, an antibiotic susceptibility detection. To this end, the variance of the nano-scale vibrations of the microcantilevers is analysed, wherein an increase of the variance evidences the vibration or motion induced by the particle.

However, using the variance only, i.e., a single statistical parameter, does not in each case suffice to discriminate between different states. For instance, in a measurement recording of a susceptible strain and a resistant strain of particles being bacteria, the populations typically overlap. Therefore, a better way of separating the populations is required. Furthermore, viable and dead cells constitute an example of an extreme difference in the metabolic activity, wherein there is however also a need for a discrimination between less extreme cases or an analysis of other motional activities of particles such as a respiration level, a growth rate, etc.

### SUMMARY OF THE INVENTION

It is an object of the present invention to provide a method that enables an improved analysis of the motional activity of particles. In particular, it is an object to provide a method that enables an improved analysis of the motional activity of particles in similar states or under complex conditions.

This object is achieved with the method according to claim 1. That is, a method of analysing a motional activity of particles with a motion detector is provided. The motion detector comprises a flexible support being configured to deflect such as bend, a detection device for detecting signals being generated upon the deflection of the flexible support, and an evaluation device for evaluating the detected signals. The method comprises the steps of i) bringing at least one particle into contact with the flexible support, ii) detecting at least one time-dependent signal that is indicative of deflections of the flexible support due to motions of the at least one particle, using the detection device, and iii) evaluating the detected time-dependent signal with the evaluation device. It is preferred that steps i) to iii) are consecutively and in said sequence performed. The evaluation of the detected time-dependent signal comprises a) analysing a time-dependency of the signal to derive a plurality of signal parameters from the signal that characterise a variation of the signal as a function of time, and b) executing a linking algorithm that has, as input variables, an input vector comprising at least a selection of said signal parameters, and has, as an output variable, at least one activity indicator that is indicative of a motional activity of the particle. It is preferred that steps a) and b) are consecutively and in said sequence performed, i.e. step b) is preferably performed after step a).

The present invention is based on the insight that the particles being in contact with the flexible support cause random signals, i.e. signals that vary in time but do not follow a simple oscillation pattern so as to appear, on first sight, to be random, or stated differently, signals being buried in noise background, measurement artefacts and the like, while comprising useful information on the motional activity of the particle, and wherein said information can be extracted using mathematical methods. In fact, the present method enables an extraction of said information by analysing the changes of the signal properties in time. That is to say, in the method according to the invention the time-dependent signal is analysed using mathematical methods. The mathematical methods can be applied to the detected signal in the time domain and/or to transformed signals obtained by transforming the detected signals into the frequency domain. In any case, the signal is detected for a plurality of points in time so as to obtain a time-dependency of the signal, and the time-dependency is analysed in order to determine time-independent signal parameters. These signal parameters characterise the time variation of the signal. At least a selection of these signal parameters is then fed to the linking algorithm as the input variables, and the linking algorithm provides at least one output variable being based on the input variables and being at least one activity indicator being indicative of a motional activity of the particle.

The flexible support can be a cantilever, preferably an AFM cantilever, a fibre such as a hollow fibre or a glass fibre, a membrane, a wire, a sponge, a flexible electrode, etc. It is furthermore preferred that the particles are attached to a surface of the flexible support before being analysed. Said attachment is preferably done according to methods as known in the state of the art such as by functionalizing the surface of the flexible support or by using attachment compounds known in the art, respectively. It is particularly preferred that the particles are attached to the surface of the flexible support as described in WO 2021/130339, which is incorporated herein by reference. That is, it is preferred that the particles are dispersed in a solution comprising at least one of a gelling agent, a gellable agent and a thickening agent, and wherein said dispersion is subsequently added to the surface of the flexible support.

Moreover, the motion detector preferably corresponds to the nanoscale motion detector as disclosed in WO 2013/054311, which is incorporated herein by reference as well. Hence, the deflections of the flexible support are preferably measured by an optical level detection method, wherein changes of position of a spot caused by laser light being reflected from the surface of the flexible support are measured with an optical detector such as a photodetector, particularly preferably with a position sensitive photodetector. The changes of position of the spot cause changes of signal recorded by the detector. As will be explained in greater detail further below, said signal is preferably processed before the signal parameters are derived from it. As such, said signal can also be referred to as a raw input signal (RIS).

The method preferably comprises, in the step of analysing the time-dependency of the signal, defining a plurality of time intervals, wherein the time-dependency of the signal is analysed within each time interval separately in order to obtain a value for each of a plurality of signal estimators for each time interval, and wherein a time series of each signal estimator is analysed in order to obtain each of the signal parameters.

The signal estimators can be determined in the time-domain or in the frequency-domain. In the latter case, it is preferred that the power spectra are estimated for each time interval of the signal.

Each signal estimator value is preferably determined by fitting a noise model to the power spectrum of the signal and/or by applying at least one statistical algorithm to the signal. The signal estimators are preferably estimators of moments of probability density functions and/or geometrical properties of power spectral density functions and/or percentiles of probability density functions and/or correlation parameters and/or partial correlation parameters and/or parameters of auto-regressive moving average models and/or parameters of nonlinear auto-regressive moving average models.

The noise model is preferably selected from a flicker noise model, a white noise model, a band noise model or combinations thereof. The Flicker noise model is preferably expressed as the weighted sum of 1/f^{α} functions, wherein f means the frequency and alpha (a) is a free parameter. The white noise model is preferably expressed as a constant or constant being modulated by frequency responses of the motion detector. The band noise model is preferably expressed as a rational function of an order being preferably optimised for a particular application. For instance, the second order band noise model could correspond to the damped harmonic oscillator-based model in susceptibility tests of E. coli strains. Examples of geometrical properties of power spectral density functions are extreme points, inflection points, the area under different parts of the curve, or horizontal and vertical distances between them.

Regarding the estimators of moments of probability density functions, it should be noted that they can have different orders of moments, they can be standardised or not, etc. For instance, the estimators of moments of probability density functions can be the variance, skewness, kurtosis, etc. It is furthermore preferred that they are optimised for particular applications, e.g., for E. coli antibiotic susceptibility detection the variance could be utilised. Regarding the percentiles of probability density functions, it should be noted that they can have different percentile ranks. For instance, the percentile rank could be the 25th percentile, the 50th percentile (median), the 75th percentile, etc. It is furthermore preferred that the percentile rank is optimised for particular applications, e.g., for metabolic activity classifications the median could be used.

As has been outlined above, the signal analysis can be based on signal parameters being related to different noise models, wherein the signal estimators are determined by fitting a noise model to the power spectral density function of the signal, for instance. However, these theoretical models are as useful as practice follows theory. In order to properly analyse cases where estimated spectra diverge from theoretical models, the geometrical properties of the spectrum are helpful.

Therefore, analysing the time-dependency of the signal using power spectral density functions is a useful method to analyse random signal properties in the frequency domain.

Hence, in the method according to the invention, each signal estimator can be a geometrical property of a power spectral density function.

Various methods of determining the power spectral density function are conceivable, wherein these methods are well-known in the art. For example, the power spectral density function can be determined by the Welch's method.

The geometrical property can be an extreme point of the power spectral density function, an inflection point of the power spectral density function, an area under a curve described by the power spectral density function or a horizontal distance and/or a vertical distance between them. Examples of an extreme point of the power spectral density function are a global maximum or a local maximum or a global minimum or a local minimum of the power spectral density function. The inflection point of the power spectral density function is understood as the point where the power spectral density function changes direction of its curvature. The area under a curve described by the power spectral density function is understood as an area under the power spectral density function between two points on the power spectral density function. Said area is preferably determined by integrating the power spectral density function between said two points. To this end, any two points on the power spectral density function can be used. Horizontal and/or vertical distances between them are, for example, horizontal and/or vertical distances between extreme points, or between inflection points, etc.

Signal parameters that are derived from such signal estimators can be seen as spectral signal parameters. Quantile signal parameters are a generalisation of spectral signal parameters. As has just been outlined above, spectral signal parameters can be calculated on the basis of the power spectral density function. The power spectral density function of the signal is estimated as an average of signal periodograms, wherein a periodogram is the squared magnitude of a Fourier transform of a signal segment. The power spectral density function is statistics. It means that it reveals the properties of a random signal (stochastic process). If the average of periodograms is used then some sporadic but high amplitude events deteriorate this statistic. For this reason, very frequently robust statistics are used like median for instance. However, if these sporadic events carry on useful information, not only median but the probability distribution of periodograms has found to be useful in order to capture said information.

Hence, in the method according to the invention the at least one signal estimator can be obtained by determining, for each time interval, a plurality of periodograms. For all periodograms, at least one quantile for at least one frequency range is determined. The quantile preferably is a percentile. These signal parameters can be referred to as quantile signal parameters.

The quantiles, in particular the percentiles, can be determined for various frequency ranges. For instance, the frequency ranges for which the quantiles or percentiles of the periodograms are estimated can be 0-10 Hz, 10-100 Hz, etc., although other frequency ranges are likewise conceivable. In other words, for each periodogram, quantiles such as percentiles for particular frequency ranges are determined. As such, a set of time series of signal estimators being quantiles of periodograms, in particular being quantiles such as percentiles for specified frequency ranges, are obtained.

This set of time series can be extended, and wherein the at least one signal parameter is obtained from the extended time series. In other words, the time series of the signal estimators can be extended by combining time series of signal estimators, and wherein the signal estimators are obtained from the combined time series.

In particular, the time series of at least one of the signal estimators can be extended so as to form an extended time series by combining at least two signal estimators into differences and/or ratios thereof.

Additionally or alternatively, the time series of at least one of the signal estimators can be extended so as to form an extended time series by transforming a plurality of quantiles, in particular percentiles, into values of an empirical probability density function. In particular, all quantiles such as all percentiles can be transformed to values of an empirical probability density function (EPDF), and wherein two consecutive quantiles such as percentiles are used to calculate one EPDF value. Furthermore, at least one distance between the values of the empirical probability density function and values of a theoretical probability density function can be determined. For instance, the distance can be measured between all EPDF values and theoretical values of a Chi2 probability density function. Examples of distances are Kullback-Leibler divergence and Jensen-Shannon distance, although other distances are likewise conceivable. Additionally or alternatively, at least one geometrical property of the empirical probability density function can be determined and/or various moments of EPDF (mean, variance, skewness, kurtosis, etc.)

At least one signal parameter can be derived from the time series of at least one of the signal estimators and/or from the extended time series of at least one of the signal estimators by i) defining a plurality of sub-series for the time series and/or for the extended time series and, for each sub-series, determine statistical properties and preferably furthermore at least one of ratios, differences and ratios of differences thereof, and/or ii) fitting one or more parametrized curves comprising one or more parameters to the time series and/or to the extended time series, and/or iii) defining a plurality of sub-series for the time series and/or for the extended time series and, for each sub-series, fit one or more parametrized curves comprising one or more parameters in order to obtain fitted parameters and preferably furthermore determine ratios, differences and ratios of differences thereof.

Conceivable statistical properties are percentiles and moments, for instance. Conceivable curves are exponential curves, polynomial curves, trigonometric curves, and linear and nonlinear mixtures thereof.

The power spectral density function of the signal of certain particles revealed a strong 1/f characteristic. This kind of spectrum is often connected with signal self-affinity. Self-affinity can be analysed by detrended fluctuations analysis (DFA) and its generalisation multifractal detrended fluctuations analysis (MF-DFA). In these methods, the dependence of statistical properties of the signal on the time scale is investigated.

Hence, in the method according to the invention, the evaluation of the detected time-dependent signal with the evaluation device can comprise the detection of the time-dependent signal during a time t, and wherein analysing the time-dependency of the signal preferably comprises the steps of:
Dividing the signal of time t into time intervals of length T, and
For each time interval j=1, . . . t/T, performing the steps of:
   calculating the digital integral of the signal within each time interval;
   divide the time interval into at least one number of subintervals;
   for each subinterval, detrend the digital integral of the signal by applying a detrending algorithm to the digital integral of the signal;
   for the at least one number of subintervals, combine the subintervals into a detrended interval; and
   determine at least one generalized mean of the detrended signal of the detrended interval for the at least one number of subintervals, whereby at least one signal parameter and/or initial signal parameter is obtained.

The digital integration is applied to the signal within every time interval. As a result, the signal within every time interval becomes an integrated signal. The digital integration of any digital signal xᵢ of N samples can be defined by the following equation

$$for every n = 1 … N , {y}_{n} = {\sum }_{i = 1}^{n} {x}_{i}$$

The detrending algorithm preferably fits a polynomial of any order (i.e. the second order) to the integrated signal and further subtracts the fit from the integrated signal. That is, the fit residue becomes the detrended integrated signal. The detrending is preferably applied to every subinterval separately. As a result all subintervals become detrended subinterval.

The integrated and detrended signals for all subintervals for the specific number of subintervals the time interval is divided into are combined back into the one signal for the whole time interval (e.g. 20 min interval of the signal is integrated and after that divided into 4 5 min subintervals, each of them is detrended and all are combined back into 20 min interval signal). The result of detrending depends on the number of subintervals the signal time interval is divided into. That is, it depends on the length of each subinterval.

At least one generalized mean of a signal time interval (integrated, detrended for at least one number of subintervals and combined back) is calculated (e.g. F₂(128) means generalized mean with exponent 2 and 128 subintervals).

Generalized mean with non-zero exponent/power q of any digital signal xᵢ of length N is defined by the following equation:

$${F}_{q} = {( \frac{1}{N} {\sum }_{i = 1}^{N} {\vert {x}_{i} \vert}^{q} )}^{\frac{1}{q}}$$

Generalized mean with zero exponent/power q of any signal xᵢ of length N is defined by the following equation:

$${F}_{0} = exp ( \frac{1}{N} {\sum }_{i = 1}^{N} ln ( {x}_{i} ) )$$

The generalized mean with exponent 2 is simply standard deviation, with exponent 0 it is geometrical mean.

The signal parameter is preferably obtained from generalized means and/or their ratios and/or differences.

In particular, signal parameters can be derived from other signal parameters such as by combining two or more signal parameters into ratios and/or differences thereof, wherein the resulting signal parameters become independent of certain factors such as environmental changes.

The method preferably further comprises executing a feature selection algorithm to automatically select a subset of signal parameters so as to obtain the input vector being fed to the linking algorithm. The feature selection algorithm is preferably configured to select the subset of signal parameters in such a manner that the subset is optimal to a multi-objective criterion.

Preferred feature selection algorithms implement a univariate selection such as F-test, mutual information, chi-squared, a penalty based method such as L1, L2 and elastic net, and forward or backward feature selection based on properties of particular linking algorithm (like coefficients in logistic regression or importances in tree-based algorithms) or based on various forms of cross-validation or bootstrapping with multi-objective criterion.

The multi-objective criterion preferably is at least one of an accuracy, a sensitivity, a specificity, a mean squared error, a mean absolute error, a root mean square error, a fraction of variance unexplained, a coefficient of determination, a number of signal parameters or a generalisation gap.

For instance, the input vector can comprise signal parameters being optimal in the Pareto sense. Being optimal in the Pareto sense is understood as being a non-dominated solution for which an improvement of all criteria is not possible. In other words, improving one criterion would deteriorate at least one other criterion. As an example, when the feature selection algorithm corresponds to the forward selection with cross-validation algorithm, a selection of the signal parameter could occur when cross-validation scores are not improved in the Pareto sense. Additionally or alternatively the input vector can comprise signal parameters being optimal with regard to a single criterion being comprised of a linear combination of two or more, for instance of all criteria. Using a linear combination of two or more criteria can be understood as to capture several aspects of the assessment of the linking algorithm. Additionally or alternatively, the input vector can comprise signal parameters being optimal with regard to one of the criteria mentioned above however under constraints, for instance constraints being defined by experts in the field of the art or on other criteria such as the specificity not being lower than 80%. These constraints can be defined after an analysis of the Pareto optimal solutions.

A drift cancellation is preferably applied to the signal prior to the derivation of the signal parameters from the signal. That is, the drift cancellation is preferably applied to the raw input signal, or RIS, mentioned initially. Again in other words, the signal estimators are preferably determined after the drift cancellation.

The drift cancellation preferably comprises a curve-fitting procedure that models a drift component of the signal or a high-pass filtering. Said drift cancellation serves the purpose of cancelling a drift of the motion detector that manifests itself in the detected signals. Said drift can be caused by various factors such as the temperature, humidity, change of state of a surrounding fluid, etc.

For instance, a drift cancellation can be performed by splitting the signal, in particular the RIS, into intervals and to thereafter apply a curve fitting in order to model a drift component of the drift. Examples of curves to be fitted are polynomials, exponential functions, trigonometric functions, or their mixtures. The fitted curves can be defined in a linear and in a nonlinear form. The type of curve, the number of fitting parameters, and the length of time intervals are preferably optimised for particular applications. The fitting error is preferably used by the next steps of the analysis, in particular in the calculation of the time series of the signal estimators.

Another example of a drift cancellation comprises the application of the just described curve fitting, however in a moving window regime and with fitting parameters that are continuously adapted. The fitting error is preferably used by the next steps of the analysis, in particular in the calculation of the time series of the signal estimators.

In this example, the fitting is preferably repeated for every single point of the signal, in particular the RIS, while using a buffer such as a circular buffer storing the last samples of the signal. The fitting is preferably modified so as to efficiently update curve fitting parameters in order to be able to process millions of signal points. The type of curve, the number of fitting parameters, and the length of the buffer are preferably optimised for particular applications as well.

Another example of a drift cancellation comprises the application of a high-pass filtering to the signal, in particular, the RIS, and wherein the high-pass filtering preferably has a particular order, type, and parameters including a phase shift. Examples of the type are Butterworth, Chebyshev I and II, Kaiser, elliptic, etc. The filter output is preferably used by the next steps of the analysis, in particular in the calculation of the time series of the signal estimators.

The drift cancellation algorithm preferably is a detrending algorithm. Said detrending algorithm is preferably configured to detrend the signal.

In particular, in the event of the at least one signal parameter being a spectral signal parameter as described earlier, it is particularly preferred that a detrending algorithm is applied to the signal before the time-dependency of the signal is analysed within the time intervals.

In the event of the at least one signal parameter being determined from a signal estimator being a quantile such as a percentile of a probability density function of periodograms, it is particularly preferred that a detrending algorithm is applied to the signal before the periodograms are determined, and wherein the plurality of periodograms is determined for the detrended signal of each time interval.

For instance, the detrending algorithm can be a fitting algorithm that fits a polynomial of the kth order to the signal and subtracts the fitted polynomial from the signal, whereby the signal is detrended. By detrending the signal a drift of the signal can be removed.

The linking algorithm particularly preferably is a regression algorithm or a classification algorithm.

In the event that the linking algorithm is a regression algorithm, the multi-objective criterion mentioned earlier preferably is at least one of the mean squared error, the mean absolute error, the root mean square error, the fraction of variance unexplained, the coefficient of determination and the like, the number of signal parameters, or the generalisation gap.

In the event that the linking algorithm is a classification algorithm, the multi-objective criterion mentioned earlier preferably is at least one of the accuracy, the sensitivity, the specificity and the like.

It is furthermore preferred that the linking algorithm is a machine learning (ML) algorithm. In some embodiments, the ML algorithm may be an ML regression algorithm, specifically, an ML algorithm implementing linear regression, decision tree regression, random forest regression, gradient boosting trees regression, Kernel regression, a multiperceptron neural network, and RBF neural network regression. Such algorithms are well known in the art.

In other embodiments, the ML algorithm may be an ML classification algorithm, specifically, an algorithm implementing logistic regression, decision tree, random forest, gradient boosting trees, support vector machines, multiperceptron neural networks, and Radial Basis Functions (RBF) neural networks. Also these types of algorithms are well known in the art.

Many other suitable ML algorithms are known in the art and may be employed.

The particle preferably is a biological object and/or a part thereof and/or a non-biological object and/or a part thereof. A preferred biological object is a cell such as a prokaryotic cell or an eukaryotic cell, a spore, a virus, a phage, and a matter of biological origin such as vesicles, peptides, proteins, polysaccharides, lipids, glucides, nucleic acids and co-polymers, protein-RNA co-polymers, RNA-DNA co-polymers, protein-DNA co-polymers, RNA/DNA-protein co-polymers, protein-protein co-polymers, or capsules. A preferred part of a biological object is an organelle. A preferred non-biological object is a protein, lipid, nucleic acid such as DNA, and nanodevices. The cell preferably is a living cell and/or the organelle preferably is a living organelle. The cell can be a prokaryotic cell and/or a eukaryotic cell. The cell can also be a motile cell and/or a non-motile cell. The cell preferably is a bacterial cell and/or a fungal cell such as a yeast cell, and/or mammalian cell and/or insect cell and/or plant cell. The organelle preferably is a mitochondrium and/or a nucleus or any other subcellular structure.

The activity indicator is preferably indicative of at least one of a metabolic activity of the particle, a physiological state of the particle, a respiration level of the particle, an ATP level of the particle, a ratio of NADH/NAD⁺ of the particle, a membrane potential of the particle, a growth rate of the particle, a kill rate of the particle, an interaction of the particle with other particles, and an interaction of the particle with an environmental factor of the particle or a combination of any of those.

The environmental factor can be of a chemical nature, e.g., a metabolite or a drug such as an antibiotic, a toxin, a denaturing agent or any other chemical compound affecting the activity of the particle. The environmental factor can however likewise be of a physical nature, e.g., a temperature, humidity, viscosity, oxygen level, redox potential, radiation, pressure or any other impact affecting the activity of the particle. The interaction of the particle with the environmental factor can be understood as a response of the particle to the environmental factor. The metabolic activity of the particle can be a respiration, energy household (e.g., ATP, NADH, NAD+ levels), membrane potential, activity of ion channels, activity of the cytoskeleton, etc. The physiological state of the particle can be a response to one or more of the aforementioned environmental factors. The interaction of the particle with other particles could be a killing, proliferation, predation, mating, quorum sensing, or conformational changes of membrane complexes and/or protein complexes and/or other cellular complexes and/or macromolecular complexes.

The particle is preferably subjected to at least one chemical stimulus and/or at least one physical stimulus. The chemical stimulus preferably is an addition of a drug such as antibiotics or a compound affecting the metabolism or viability of the particle such as the cell. The chemical stimulus can likewise be a change in an environmental condition such as a culture condition, etc. The physical stimulus preferably is an application of stress.

The activity indicator is preferably determined before and/or during and/or after the particle is subjected to the chemical stimulus and/or the physical stimulus. It is furthermore preferred that the thus determined activity indicators are compared with one another.

For instance, if one were to analyse the effectiveness of a drug on particles such as antibiotics on bacteria, the activity indicator could be determined before the addition of the antibiotics to the bacteria and after the addition of the antibiotics to the bacteria. If the antibiotics are effective, one would determine a difference in the activity indicators. For instance, the killed bacteria would no longer exhibit a metabolic activity. In addition, dying or susceptible bacteria can alter their metabolic activity upon the exposure to an antibiotic. That is, the bacteria must not immediately be dead.

In a further aspect, a method of generating a training data set of a machine learning algorithm is provided. Said method comprises the steps of:
a) Bringing at least one particle into contact with a flexible support,
b) Detecting at least one time-dependent signal that is indicative of deflections of the flexible support due to motions of the at least one particle, using a detection device, and
c) Evaluating the detected time-dependent signal with an evaluation device, wherein the evaluation of the detected time-dependent signal comprises analysing a time-dependency of the signal in order to derive a plurality of signal parameters from the signal that characterise a variation of the signal as a function of time,
d) Performing an independent measurement in order to derive at least one associated activity indicator that is indicative of a motional activity of the particle,
e) Saving the plurality of signal parameters and the at least one associated activity indicator in the training data set, and
f) Repeating steps a) to e) for a plurality of particles exhibiting different motional activities.

The training data set thus comprises a plurality of the signal parameters and the associated activity indicators for a plurality of particles.

In a further aspect, a method of training a machine learning algorithm is provided, wherein the machine learning algorithm is trained with the training data set mentioned above.

That is, the machine learning algorithm is trained with the signal parameters and the associated activity indicators. Consequently, the machine learning algorithm can make predictions of activity indicators based on signal parameters and vice versa without knowledge of the particles and their motional activity being necessary.

For instance, the training can be based on a training data set comprising experimental results being detected by the detection device of a motion detector. This training data set is then preferably labelled by results of reference experiments that measure, for instance, the metabolic activity of interest using a reference method such as the susceptibility of strains being measured by the Kirby-Bauer test. Said labelled data is then used to select the signal parameters, optimise all stages of the linking algorithm and assess performance using cross-validation techniques.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows a schematics of the preferred steps of the method according to the invention;

FIG. 2 shows a signal being detected with a motion detector that has been subject to a drift cancellation during evaluation with the method according to the invention;

FIG. 3 shows a signal being detected with a motion detector that has been subject to the fitting of noise models in the determination of signal estimators during evaluation with the method according to the invention;

FIG. 4 shows a graph depicting the accuracy of various algorithms for various numbers of signal parameters in order to identify Pareto optimal feature selection algorithms suitable for the method according to the invention;

FIG. 5a shows a schematics of a motion detector comprising a cantilever with attached viable bacteria (upper part) and a time series of the signal estimator that has been determined during the evaluation with the method according to the invention (lower panel);

FIG. 5b shows a schematics of a motion detector comprising a cantilever with attached dead bacteria (upper part) and a time series of the signal estimator that has been determined during the evaluation with the method according to the invention (lower panel);

FIG. 6a shows a graph depicting the signal estimators that have been determined during the evaluation depicted in FIGS. 5a and 5b;

FIG. 6b shows a graph depicting the fluorescence over time that has been determined for the viable bacteria and the dead bacteria used for the analysis depicted in FIGS. 5a and 5b;

FIG. 7a shows a graph depicting a signal being detected with the motion detector for an E. coli strain ATCC-25922 under different conditions and for which a signal estimator over time that has been determined during the evaluation with the method according to the invention;

FIG. 7b shows a graph depicting a signal being detected with the motion detector for an E. coli strain BAA-2452 under different conditions and for which a signal estimator over time that has been determined during the evaluation with the method according to the invention;

FIG. 8a shows a graph depicting further signal parameters that have been determined during the evaluation with the method according to the invention for the signals being detected with the motion detector for an E. coli strain ATCC-25922 and an E. coli strain BAA-2452 under different conditions;

FIG. 8b shows a graph depicting a linear combination of the further signal parameters of FIG. 8a;

FIG. 9 shows a graph depicting a linear combination of further signal parameters that have been determined during the evaluation with the method according to the invention for the signals being detected with the motion detector for an E. coli strain ATCC-25922 and an E. coli strain BAA-2452 under different conditions;

FIG. 10 shows a graph depicting the signal estimator and the signal parameters—medians of the time series of the signal estimator that have been determined during the evaluation with the method according to the invention for a signal being detected with the motion detector for a NARA-5032 resistant E. coli strain under different conditions;

FIG. 11 shows a graph depicting the determination of signal parameters by linear and exponential fitting to the signal estimator depicted in FIG. 10;

FIG. 12 shows a table depicting the performance of a feature selection algorithm suitable for the evaluation with the method according to the invention for different input vectors;

FIG. 13a shows a graph depicting the correlation between signal parameters of the method according to the invention and a kill rate determined with standard microbiological methods;

FIG. 13b shows a graph depicting the correlation between other signal parameters of the method according to the invention and a kill rate determined with standard microbiological methods;

FIG. 13c shows a graph depicting the correlation between a linear combination of the signal parameters of FIGS. 13a and 13b and a kill rate determined with standard microbiological methods;

FIG. 14a shows a graph depicting a time series of a signal estimator being determined in the method according to the invention for E. coli ATCC-25922 under different conditions;

FIG. 14b shows a graph depicting a time series of a signal estimator being determined in the method according to the invention for E. coli ATCC-25922 under different conditions;

FIG. 15a shows graphs depicting signal parameters that were determined from the time series of the graphs of FIGS. 14a and 14b;

FIG. 15b shows a graph depicting a combination of the signal parameters that were determined from the time series of the graphs of FIGS. 14a and 14b;

FIG. 16a shows a graph depicting a linear combination of further signal parameters that were determined from the time series of the graphs of FIGS. 14a and 14b;

FIG. 16b shows a graph depicting the ATP (adenosyl-triphosphate) concentration, a marker for metabolic activity, of E. coli ATCC-25922 under culture conditions being measured in the graphs of FIGS. 14a and 14b;

FIG. 17 shows a schematic of a motion detector;

FIG. 18a shows the distribution of Klebsiella pneumoniae strains according to their reference MICs for ciprofloxacin (CIP);

FIG. 18b shows the performance of the linking algorithm being a classification algorithm depending on the number of signal parameters (SPs);

FIG. 19 shows the performance of classification models being linking algorithms with different numbers of SPs for Klebsiella pneumoniae and ciprofloxacin;

FIG. 20a shows the score of each experiment used to train Model 1 of FIG. 19 using one SP plotted against the reference MIC for ciprofloxacin;

FIG. 20b shows the score of each experiment used to train Model 4 of FIG. 19 using four SPs plotted against the reference MIC for ciprofloxacin;

FIG. 21a shows the distribution of E. coli and Klebsiella pneumoniae strains according to their reference MICs for ceftriaxone (CRO);

FIG. 21b shows the score of each experiment used to train the CRO Model in FIG. 24 plotted against the reference MIC for ceftriaxone;

FIG. 22a shows the distribution of E. coli strains according to their reference MICs for ciprofloxacin (CIP);

FIG. 22b shows the score of each experiment used to train the CIP Model in FIG. 24 plotted against the reference MIC for CIP;

FIG. 23a shows the distribution of E. coli strains according to their reference MICs for cefotaxime (CTX);

FIG. 23b shows the score of each experiment used to train the CTX Model in FIG. 24 plotted against the reference MIC for CTX;

FIG. 24 shows the performance of classification models for data shown in FIGS. 21b (CRO), 22b (CIP) and 22c (CTX);

FIG. 25ab shows a graph depicting a signal being detected with the motion detector for a cancer cell line SW480 under different conditions (a, with doxorubicin and b, media control) and for which a signal estimator over time that has been determined during the evaluation with the method according to the invention;

FIG. 26 shows an illustration of geometrical properties of the power spectral density function of vibrations. They are used to calculate described integrals N1-N5, distances and ratios. Part from 20 to 200 Hz is used to fit line to log-log plot to determine N0 and alpha (slope and intercept of log-log line fit);

FIG. 27 shows a comparison between quantile spectrum and power spectral density. The 50th percentile spectrum (P50) is close to the classic power spectral density function (PSD). Other percentiles introduce additional information about probability distribution of Fourier components;

FIG. 28 shows the empirical probability density function (EPDF) calculated on the basis of spectral percentiles for three Fourier components 10, 100 and 1000 Hz. The EPDF is Chi squared distribution provided that transformed noise is Gaussian but estimation of this statistic for cell vibrations signal revealed its discrimination power;

FIG. 29 shows the MF-DFA signal parameters used in the embodiments. They are calculated as a ratio of F_{q}(number of subintervals) for the last 20 min of the drug phase and the first 20 min of the drug phase. So the signal parameter “F0” is F₋₁₀(1024) of the last 20 minutes of the drug phase divided by F₋₁₀(1024) of the first 20 minutes of the drug phase;

FIG. 30 shows the dependence of the performance of the linking algorithm being a classification algorithm on signal parameters selected by the method described in the invention in the problem of classification of metabolic response to two antibiotics: ceftriaxone and ciprofloxacin;

FIG. 31 shows the dependence of the performance of the linking algorithm being a classification algorithm on signal parameters selected by the method described in the invention in the problem of classification of metabolic response the physical stressors—temperature;

FIG. 32 shows the dependence of the performance of the linking algorithm being a classification algorithm on signal parameters selected by the method described in the invention in the problem of classification of colon cancer cells metabolic with and without presence of antibiotic;

FIG. 33 shows the results of the application of the linking algorithm being a linear regression algorithm with quantile signal parameters for the prediction of metabolic response to varying drug concentration. The high value of coefficient of determination indicates strong dependence between linking algorithm output and actual values of drug concentration;

FIG. 34 shows the dependence of the performance of the linking algorithm being a regression algorithm on a number of signal parameters selected by the method described in the invention in the problem of estimating quantitative metabolic response to increasing concentration of antibiotic.

### DESCRIPTION OF PREFERRED EMBODIMENTS

Various aspects of the invention shall now be illustrated with reference to the figures.

As mentioned earlier, the present invention enables an improved analysis of the motional activity of particles with a motion detector 1 comprising a flexible support 2 being in contact with the particles to be analysed and being configured to deflect due to motions of the particles, see FIG. 17. As further follows from FIG. 17, the motion detector 1 comprises a source of radiation such as a laser 5 that is irradiating electromagnetic radiation onto the flexible support 2 and furthermore comprises a detection device 3 that detects signals in the form of reflected electromagnetic radiation and being generated upon the deflection of the flexible support 2 as well as an evaluation device 4 that evaluates the detected signals and ultimately outputs at least one activity indicator that is indicative of a motional activity of the particle. In the depicted example, the motion detector corresponds to a motion detector as described in WO 2013/054311.

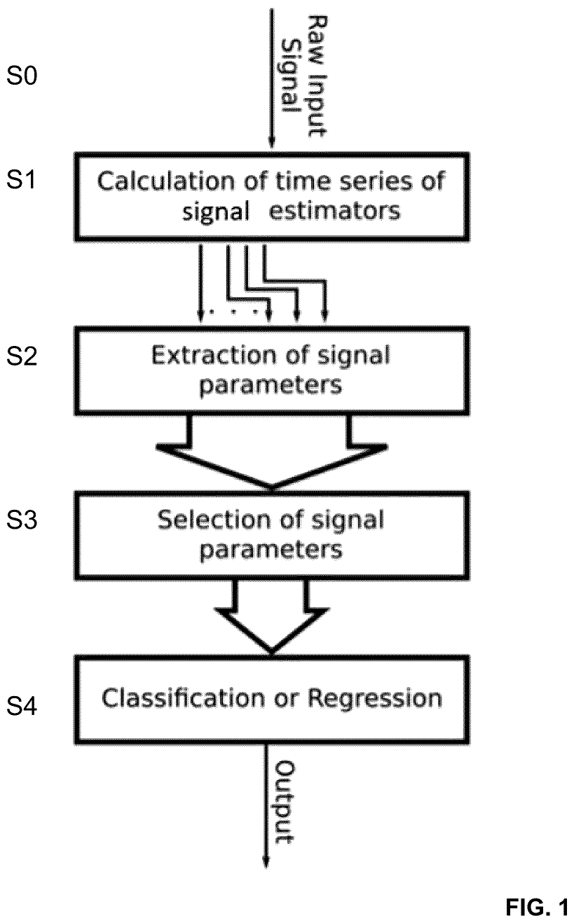

For illustrative purposes only, preferred steps as well as a preferred ordering of these steps that allow the analysis of the motional activity of the particles according to the method of the invention are summarised hereinbelow and with reference to FIG. 1.

S0: The starting point of the analysis is at least one time-dependent signal being indicative of the deflections of the flexible support of the motion detector. Here, said signal is referred to as raw input signal, RIS.

In order to cancel any drifts in the RIS, the method preferably subjects the RIS to a drift cancellation.

S1: From the signal, either from the RIS but preferably after its drift cancellation, the method calculates a time series of signal estimators. The calculation of the time series of signal estimators can be done in the time domain or in the frequency domain.

If signal estimators in the time-domain are to be calculated, the signal is split into time intervals, wherein the time-dependency of the signal is analysed within each time interval separately in order to obtain signal estimators, and wherein a time series of the signal estimators is analysed in order to obtain the signal parameters. For instance, the signal estimators can be determined by using at least one of the following:
Estimators of moments of probability density functions;
Percentiles of probability density functions;
Correlations, partial correlations, or results of auto-regressive moving average (ARMA) and nonlinear auto-regressive moving average (NARMA) modelling.

The signal estimators, for instance the values of particular statistical algorithms or noise models such as time-varying noise model parameters, preferably become samples of a new time series. These new time series of signal estimators preferably consist of fewer points, reveal characteristic patterns, and can be used as input for a determination or an extraction of the signal parameters, see further below.

If signal estimators in the frequency-domain are to be calculated, the signal is preferably split into intervals for which power spectra are estimated. The method of estimation and windowing is preferably optimised for particular applications. After that, various noise models are preferably fitted to a given spectrum and the signal estimators, here the fitting parameters, are preferably used as samples of new time series. Apart from signal estimators being parameters of noise models, also geometrical properties of the estimated spectrum could be measured (e.g. extreme points, inflection points, area under different parts of the curve, horizontal and vertical distances between them).

The result is a set of time series, each one for a signal estimator being a particular fitting parameter or property. Also in this way, the number of points can be significantly reduced, allowing identification of patterns or shapes of the time series characteristic for a given metabolic activity by the extraction of signal parameters, see further below.

S2: After the calculation of the time series of signal estimators, the times series of the signal estimators in the time-domain or in the frequency-domain are transformed to vectors of signal parameters. The transformation is preferably done by means of one of the following three methods.
The estimated time series are split into intervals. The statistical properties such as percentiles or moments are calculated for each interval. They form a vector of basic signal parameters. After that, ratios, differences, and ratios of differences of the basic signal parameters can be utilised to calculate vectors of extended signal parameters. In this way, the signal parameters become more robust to environmental conditions.
The estimated time series are fitted using curves of type and parameterization preferably being optimised for the particular application. The type of curves might be exponential, polynomial, trigonometric, and their linear and nonlinear mixtures.
The third method of extraction of signal parameters is a combination of the first two methods. To this end, it is preferred that the fitting is performed not to the whole signal but within intervals only and that a mixing using ratios and differences is performed.

The result of the extraction of the signal parameters is a large vector of real numbers (usually more than 200). This vector is preferably shortened by selecting a subset of the signal parameters by executing a feature selection algorithm.

S3: Hence, after the extraction of the signal parameters, a selection of signal parameters is performed by means of a feature selection algorithm. In this way, a robustness and a generalisation level of classification or, in a sense, prediction algorithm are provided. The feature selection algorithm selecting the signal parameters is preferably optimised for a particular application and might be one of the following:
Univariate selection (like F-test, mutual information, chi chi-squared),
Penalty based methods (L1, L2, elastic net),
Forward or backward selection of signal parameters based on properties of a particular model or based on cross-validation or bootstrapping.

The model Pareto optimality concept can be used to select signal parameters' vectors optimal to a multi-objective criterion in forward or backward selection. The number of signal parameters should be a minimum while accuracy is a maximum. Usually, it is not possible to decrease the number of used signal parameters without decreasing accuracy. From these Pareto optimal algorithms, the final linking algorithm comprising the optimal input vector of signal parameters is therefore preferably selected based on domain-specific criteria with human expert assistance, for instance. This additional criterion could be the performance for a special test dataset or additional requirements defined by an expert.

S4: The input vector obtained after the selection of the signal parameters is fed to a linking algorithm. The linking algorithm uses the input vector of signal parameters to output the activity indicator of the particles. This activity indicator may be discrete, where the input vector of signal parameters extracted and selected in the previous steps is used as an input for a classification algorithm. That is, the linking algorithm corresponds in this case to a classification algorithm. The classification algorithm can be one of the following:
Logistic regression,
Decision tree,
Random forest,
Gradient boosting trees,
Support vector machines,
Multiperceptron neural networks,
Radial basis functions (RBF) neural networks.

The type of linking algorithm and values of its hyper-parameters are preferably optimised for particular applications.

The activity indicator may also be a real number, where the input vector of signal parameters is also used as an input for a regression algorithm. That is, the linking algorithm corresponds in this case to a regression algorithm. The regression algorithm is preferably one of the following:
Linear regression,
Decision tree regression,
Random forest regression,
Gradient boosting trees regression
Kernel regression,
Multiperceptron neural network,
RBF neural network regression.

The type of linking algorithm and values of its hyper-parameters are preferably optimised for particular applications.

More practically speaking, the particles being in contact with the flexible support cause random signals buried in a significant noise background with useful information coded in both time and frequency domain. Particles such as organisms evolve in time under different stimuli (like nutrients, antibiotics, chemicals) that affect the properties of the random signals. The signal properties that provide for instance diagnostic information can be extracted using the method according to the invention by observing their changes in time under properly optimised measurement conditions, for instance. Due to the biological variability between tested organisms and between the different conditions measured, the herein proposed method has proven as a powerful tool that allows separating diagnostic information from measurement artefacts and noise background. Relating this example to the analytic outline depicted in FIG. 1, the detected signal is transformed to a time series of signal estimators coding for instance i) the changes of state of living of the organisms and ii) the changes of the state of the measurement environment itself. The time series resemble different shapes and patterns that need to be quantified and related to the phenomenon of interest. To detect a given phenomenon or to measure its intensity these time series are then subject to the extraction of the signal parameters that define here for example a single experiment and that are stable in a given application domain. To optimise the set of signal parameters in order to increase the strength of the relation between the signal parameters and the phenomenon of interest in the next stage of the analytic outline, the selection of the signal parameters is performed. Ultimately, an indicator that is indicative of the phenomenon of interest is outputted.

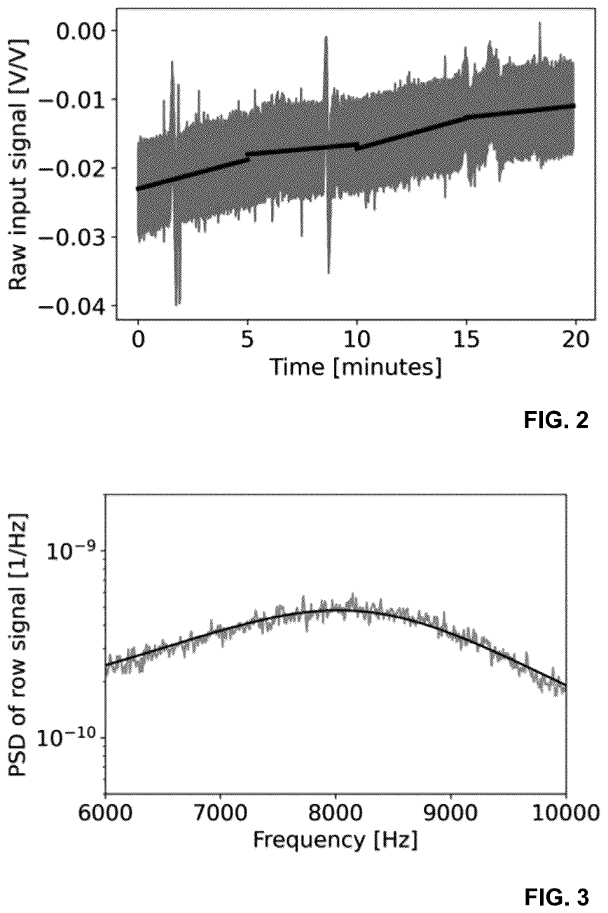

FIG. 2 depicts a signal being detected with a motion detector that has been subject to a drift cancellation during evaluation with the method according to the invention. In the depicted example the drift is cancelled by fitting a linear curve, in fact a 1^{st} order polynomial, with 5 minutes lasting intervals.

FIG. 3 depicts a signal being detected with the motion detector and that has been subject to the fitting of noise models in the determination of signal estimators. The noise model corresponds here to a mixture of white noise and band noise and comprises four fitting parameters: the amplitude of the band noise, the frequency of the poles of the band noise, the band width and the level of white noise.

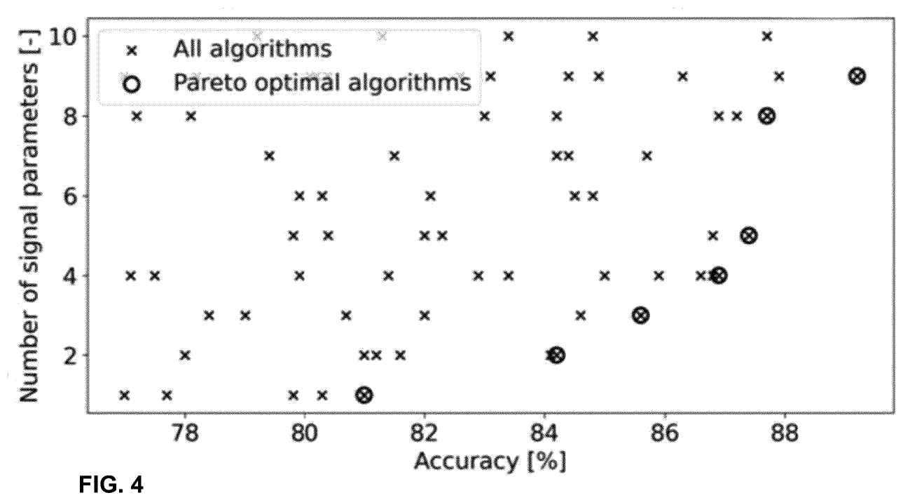

FIG. 4 illustrates the identification of Pareto optimal feature selection algorithms that could be used in the method according to the invention. The Pareto optimal feature selection algorithms are based on the accuracy and the number of signal parameters.

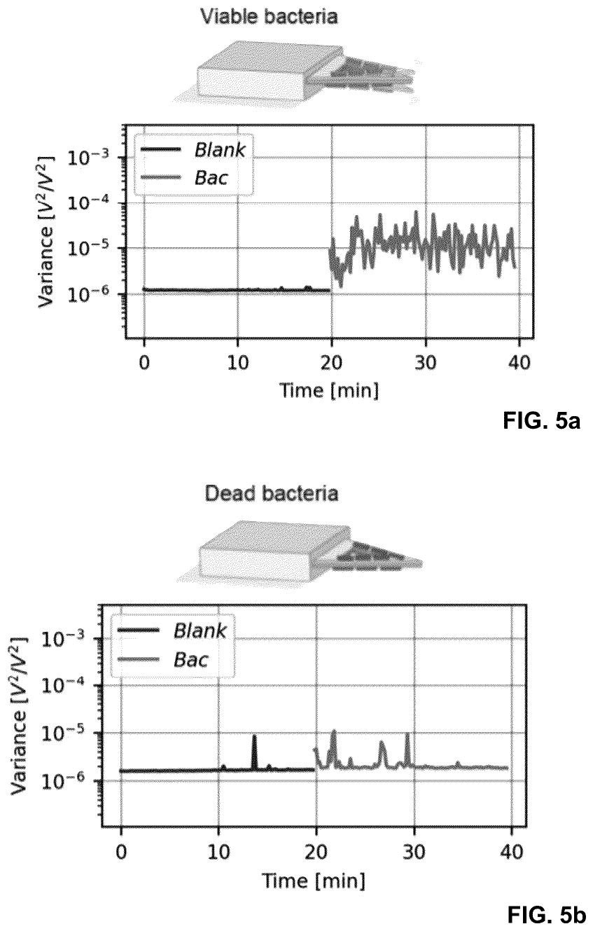

FIGS. 5a and 5b illustrate that the method according to the invention can be used for the discrimination of viable and dead cells or in broader terms cells exhibiting extreme differences in metabolic activity. This example can be considered as introductory to the method and as a very elementary and under-complex case. In particular, these figures depict in each case in the upper part a flexible support in the form of a microcantilever of a motion detector comprising viable (FIG. 5a) and dead (FIG. 5b) bacteria attached to the microcantilever.

In this experiment, the reference strain ATCC-25922 of the Gram-negative bacterium E. coli was cultured to late logarithmic phase (OD₆₀₀=1=10⁹ cells/mL) under standard laboratory conditions (i.e., nutrient-rich Miller's Luria Bertani media (LB), aerobic, 37° C.) and harvested using centrifugation (2000 g, 3′). The pellet was separated and split into two groups for the subsequent analysis. The first group of bacteria was kept alive at room temperature while the second group was exposed to 60° C. for 20 min to generate non-viable bacteria due to the denaturation of enzymatic complexes and other cellular essential processes. As expected and confirmed by an independent measurement, in this latter group, we were unable to detect metabolic activity using the redox-dependent fluorescent dye resazurin that shifts its emission/excitation spectrum due to respiratory activity, a main indicator for the metabolic state of a cell.

Prior to the start of the experiment, the microcantilever of the motion detector was functionalized with a linking agent required for attaining a sustainable cell attachment that reliably lasts throughout the nanomotion recording. The attachment was done as described in WO 2021/130339 A1. In fact, in this experimental setup, positively charged Poly-D-Lysine (PDL) was used as it can facilitate the attachment of E. coli cells exhibiting a negatively charged lipopolysaccharide surface. In fact, most cells exhibit a negative net charge on their surface and in cases where this is not the case the functionalizing agent can be adapted, see also WO 2021/130339A1. Here, a solution of 0.1 mg/ml Poly-D-Lysine in ultrapure water was applied on the microcantilever for 20 min, rinsed off again with ultrapure water and dried. Using the motion detector, the deflections of the functionalized bare microcantilever were recorded in ½ concentrated LB and served as the Blank for the subsequent measurements of the two groups of E. coli cells. Both viable and heat-killed groups of E. coli cells were then attached to a microcantilever in parallel recordings. That is, two phases of a signal recording were done: Blank phase, where the deflections of an empty cantilever are measured followed by the Bac phase where the deflections of a cantilever with attached bacteria are measured.

From the deflection of the microcantilever and after drift cancellation using linear detrending (1^{st} order polynomial) of 10s long intervals a time series of a signal estimator, in this case the variance (second moment of probability density function), is calculated and plotted for 20 min, first for the bare microcantilever (Blank phase) and second for the microcantilever with attached ATCC-25922 for each group of viable and non-viable cells (Bac phase). That is, in FIG. 5a, the variance of those deflections of the microcantilever holding immobilised E. coli ATCC-25922 in ½ LB after incubation in LB at 37° C. is shown. As just mentioned, these are viable bacteria. FIG. 5b shows the variance of the microcantilever that holds bacteria of the same strain after exposure to 60° C. for 20 min, wherein the bacteria are dead.

From the variance, i.e., the signal estimator, the signal parameter was calculated: the median of the variance over 20 min of recording for the Blank and the Bac phases. In the Blank phase the mean μ was 1.6×10⁻⁶ V²/V². After attachment of viable cells, in the Bac phase, the variance increased by one order of magnitude to 1.5×10⁻⁵ V²/V², whereas the median of the variance of heat-killed, non-viable bacteria in the Bac phase stayed at the level of the Blank phase (μ_{viable}=1.5×10⁻⁵ V²/V² vs μ_{heatkilled}=2.5×10⁻⁶ V²/V², p=0.0013, t-test, p of the variance was calculated over 20 min) in concordance with other metabolic activity assessments as, e.g., using the fluorescent dye resazurin, mentioned earlier.

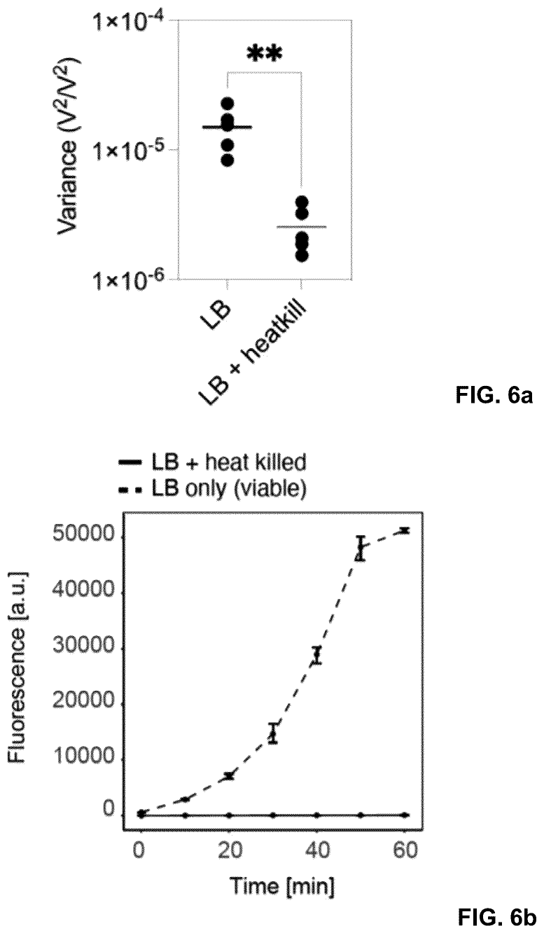

FIG. 6a depicts the median of the variance in the Bac phase of five experiments (bar represents the median, t-test p<0.005). As follows from FIG. 6a, in this extreme case of viable and non-viable bacteria, the use of one single signal parameter was sufficient for the discrimination of two metabolically different groups.

With reference to the further figures, more complex scenarios with less extreme differences between groups (in regard to biological qualities or phenotypes) are analysed by the method according to the invention.

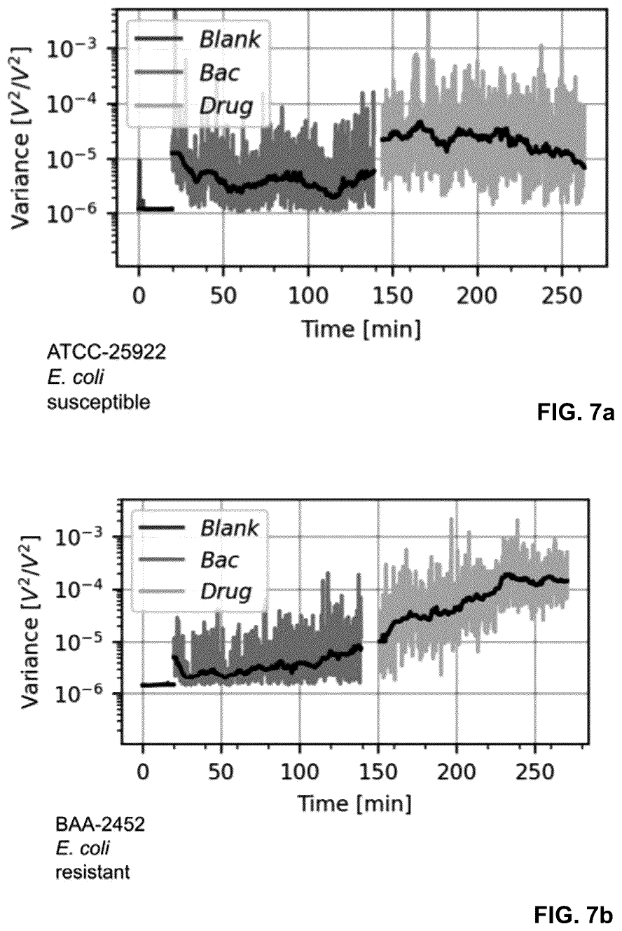

To this end, FIGS. 7a to 12 illustrate the investigation of the reaction of cells to a changing environment that can be used for classification. The changing environment corresponds here to the change from a favourable culture environment into a stress environment. In particular, these figures illustrate antibiotic susceptibility testing (AST) being performed with the method according to the invention, wherein susceptible and resistant E. coli strains are attached to the microcantilever of the motion detector and are exposed to the antibiotic ceftriaxone.

In fact, for the analysis presented in FIG. 7a, the ceftriaxone susceptible E. coli strain ATCC-25922 and for the analysis presented in FIG. 7b, the ceftriaxone resistant E. coli strain BAA-2452 was used, respectively. FIGS. 7a and 7b depict plots of the signal estimator, i.e., here the variance of the deflection of the microcantilever, over time (i), for the bare functionalized microcantilever (Blank), (ii), the microcantilever with attached bacteria in culture conditions (Bac) and (iii), for attached bacteria exposed to ceftriaxone (Drug). In the present example, ½ LB was used as culture media in the Blank and Bac phase that was supplemented with 32 μg/mL ceftriaxone in the Drug phase.

The two patterns recognizable in FIGS. 7a and 7b are characteristic for susceptible and resistant strains but the assessment is not always so clear and easy to discriminate if the analysis is not done visually by human eye but by machine.

For this reason, signal parameters were calculated and thresholded to determine if a given strain is susceptible or resistant—that is, if the value of a signal parameter is greater than a threshold value, the strain is determined as resistant, and if the value is not greater than the threshold value, the strain is determined as susceptible. To determine whether cells with different reactions to antibiotic stress can be classified, the signal of two additional susceptible clinical isolates RN-26 and RN-49, as well as of two additional resistant clinical isolates B1 and B15 from a Swiss hospital were detected with the motion detector. In total 67 experiments with these strains exposed to ceftriaxone were recorded. Signal parameters were extracted from single signal estimators that gave one of the best separations between the two different groups as for example the Median of the first 30 min of the Drug phase normalised to the Median of the entire Drug phase or the last 30 min of the Drug phase normalised to the Median of the entire Drug phase.

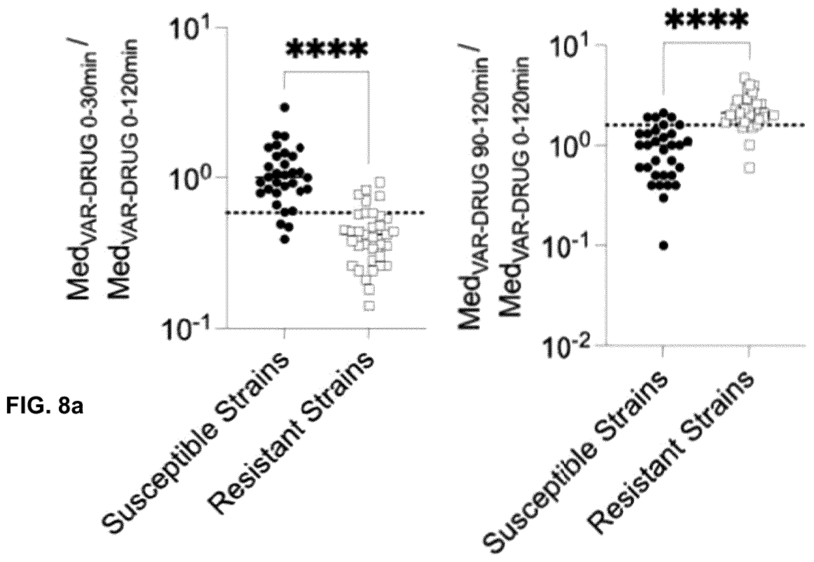

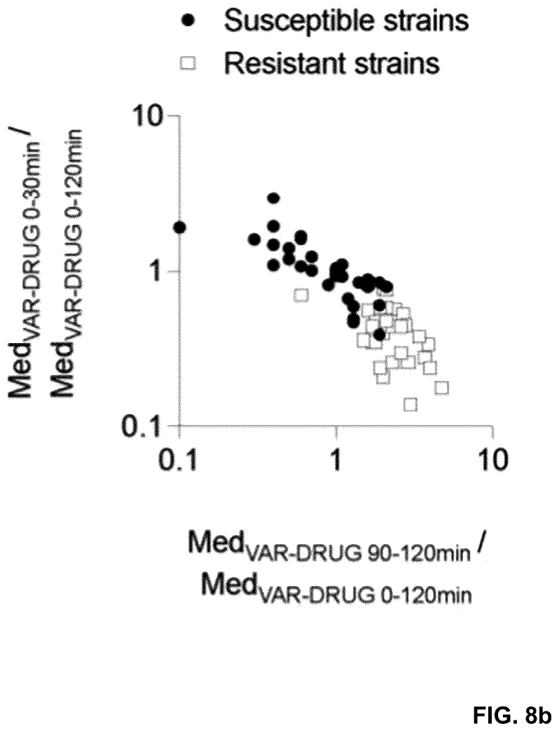

FIG. 8a depicts the two signal parameters, namely the median from the first 30 min of the Drug phase normalised to the Median of the entire Drug phase (lower graph) and the last 30 min of the Drug phase normalised to the Median of the entire Drug phase (upper graph). From these graphs, it is apparent that both signal parameters (SP) have different values for a susceptible and a resistant strain. Both examples of signal parameters show significantly different populations for a multitude of experiments using the same conditions for a number of additional susceptible and resistant strains (t-test, p<0.00005). However, using a single signal parameter does not in each case suffice to discriminate a single recording of a susceptible from a recording of a resistant strain as there is an overlap between both populations. Therefore, a better way of separation of the two populations is required. This is illustrated in FIG. 8b when separation of a susceptible and a resistant population each composed of different strains is enhanced by using both signal parameters from FIG. 8a.

From FIG. 8b it follows that the combination of signal parameters better separates two metabolic states as e.g., susceptible and resistant strains. That is, the separation of the susceptible and the resistant population each composed of different strains is enhanced by using both signal parameters from FIG. 8a. In the depicted example, the two signal parameters are expressed as a linear combination, wherein the separation line is neither vertical nor horizontal but diagonal—which mathematically is expressed as the linear combination of these two signal parameters. That is, the two signal parameters are weighted by two constants and are added together so as to form a classification indicator that might be thresholded. This approach can be generalised and a number of signal parameters can be extracted from the time series of signal estimators weighted by corresponding constants and thresholded to classify susceptible and resistant strains for which an optimal number of signal parameters would achieve the highest accuracy. The optimal weights and threshold can be estimated with a logistic regression algorithm. That is, the linking algorithm is a logistic regression algorithm here.

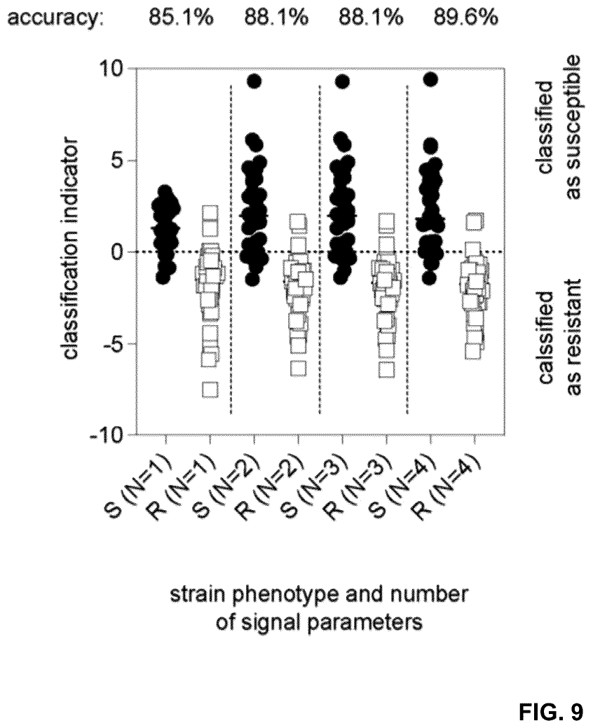

In FIG. 9 it is shown that the linear combination of more than two signal parameters (in this case four signal parameters) can further improve the separation. This linear combination of signal parameters becomes a classification indicator. That is, FIG. 9 illustrates how adding further signal parameters improves classification accuracy (percentage of correctly classified experiments). The weighted sum of signal parameters is thresholded in such a way that negative values indicate resistant strains while positive values indicate susceptible strains. In this set of 67 experiments, one signal parameter alone (ratio of median of the variance in the last 30 minutes of the drug phase over median of the variance of the whole drug phase), achieved an accuracy of 85.1%. The weighted sum of four signal parameters (median of the variance of the last 30 minutes of the drug phase over median of the variance of the whole drug phase, median of the variance of the first 30 minutes of the drug phase over median of the variance of the whole drug phase, median of the variance from 60 minute to 90 minute of the drug phase over median of the variance from 30 minute to 60 minute of the drug phase, median of the variance from 60 minute to 90 minute of the drug phase over the median of the variance for whole drug phase) increased the accuracy for the separation of both classes to 89.6%. For this limited dataset the weights and thresholds are optimised with a logistic regression algorithm to show the potential of this solution.

In practical applications however, the extraction and selection of signal parameters need to be optimised carefully. The large number of signal parameters results in so-called algorithm overfitting. In this case, high performance for the dataset used for fitting is not maintained when the algorithm is used in real conditions. For this reason, the cross-validation procedure is applied, where the dataset is split into k subsets. The algorithm is fitted on k−1 subsets and validated on the last one not used for fitting. The process is repeated k times every time with the new subset excluded. In this way the estimated mean accuracy reveals the problem of overfitting (an overfitted algorithm has worse accuracy than not overfitted).

The cross-validation helps to select only those signal parameters that handle useful information. The selection starts from one signal parameter and continuously increases the number of parameters one by one (forward selection) and after that starts to remove signal parameters (backward selection) up to a single one. The cross-validation helps to assess how addition or removal of signal parameters affects the indicator performance measured by specified single or multi-objective criterion. This procedure allows the extraction of many signal parameters from many time series of signal estimators and the selection of only the most important ones.

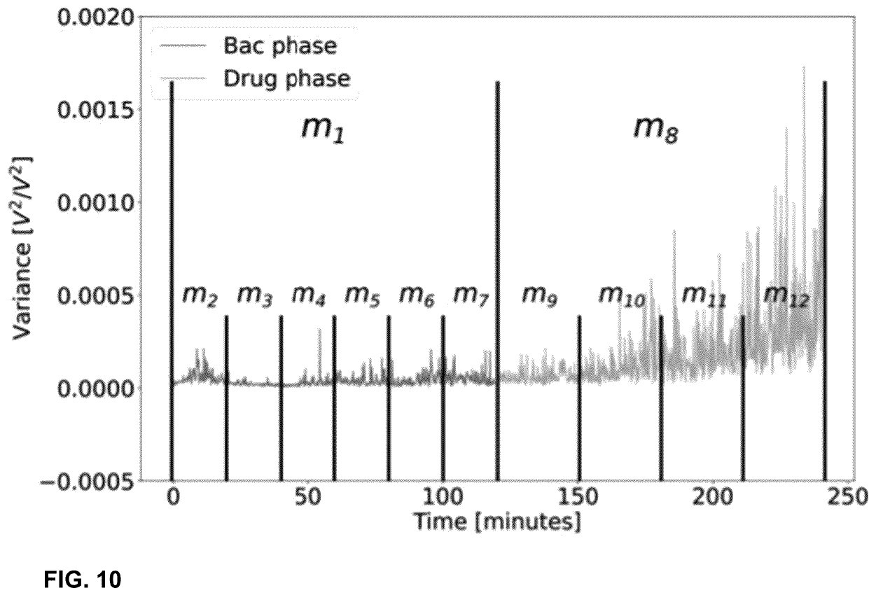

In the just presented example, a rather small dataset was used. In the following and with reference to FIGS. 10 to 12, the method of the invention is applied to a much larger dataset counting 1102 experiments with 84 clinical strains of E. coli that have been isolated from patient samples tested with ceftriaxone. 561 of those experiments were performed on susceptible strains and 541 on resistant strains.

In a first step, a drift cancellation of the detected RIS was performed. To this end the detected RIS was split into 10 s intervals. Thereafter, a linear fitting of a polynomial of the 1^{st} order to the RIS for 10 second intervals was applied to calculate the fitting error being the input for the calculation of the signal estimator, herein below of the variance (estimator of the 2^{nd} moment of probability density function). In this way the signal estimator over time, i.e. a variance signal s(t) with 0.1 Hz sampling frequency is obtained.

In the frequency domain, the 85 seconds intervals are used for power spectrum estimation and fitting by mixture of white and second order band noise models with common equation (1),

$$P ( f ) = {A [ {( {f}^{2} - {f}_{0}^{2} )}^{2} + {f}^{2} {f}_{0}^{2} {Q}^{- 2} ]}^{- 1} + B \qquad ( 1 )$$

where f means frequency, A, f₀, and Q are parameters of the band noise, and B is a parameter of the white noise (a constant). This results in additional four signals (A(t), f₀(t), Q(t) and B(t)) with sampling frequency around 0.01 Hz ( 1/85). Together with the variance signal s(t) these (time series of statistical estimators) are the input for the determination of the signal parameters.

In said determination of the signal parameters, the signal parameters are calculated. The signals s(t), A(t), f₀(t), Q(t), B(t) are divided into intervals of 20 minutes length for the Bac phase and 30 minutes for the Drug phase, see FIG. 10, depicting m₂-m₇ and m₉-m₉₂ intervals, respectively. For each interval, its median (50^{th} percentile) is calculated along with all their possible ratios. The definition of median's ratios is presented in equation (2),

$$∀ i > {jm}_{ij} = {m}_{i} / {m}_{j} \qquad ( 2 )$$

where mᵢ, mⱼ are medians within the i^{th} and j^{th} interval as defined in FIG. 10.

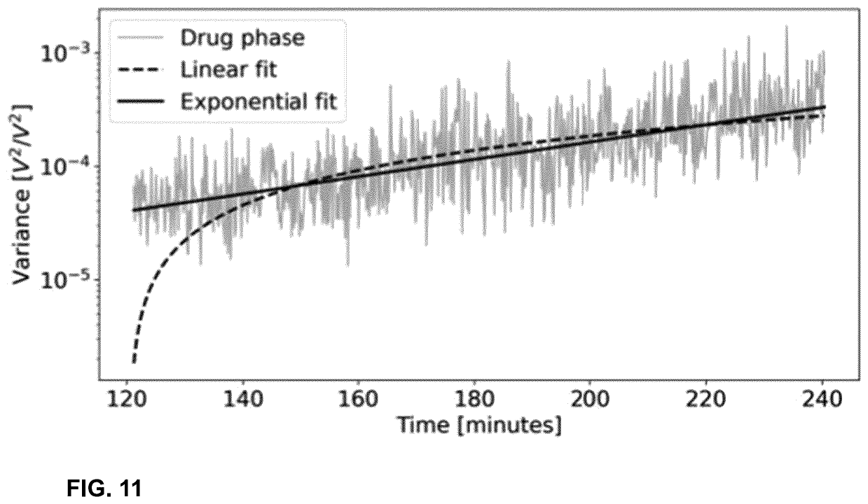

For the variance signal in the drug phase, the linear and exponential fitting are also applied, and the fitted parameters are additional four signal parameters (the slope a, the intersection b, the growth/decay rate τ, and the magnitude c). Equation (3) defines the exact mathematical formulas,

$$x ( t ) = a t + b , x ( t ) = c exp ( τ t ) \qquad ( 3 )$$

where coefficients of determination of both fits and ratios c/T and b/a are among the set of extracted signal parameters. The illustration of linear and exponential fit is shown in FIG. 11.

As a result, a set of 192 signal parameters has been determined or extracted and has been subjected to signal parameters selection stage, i.e., has been fed to the feature selection algorithm.

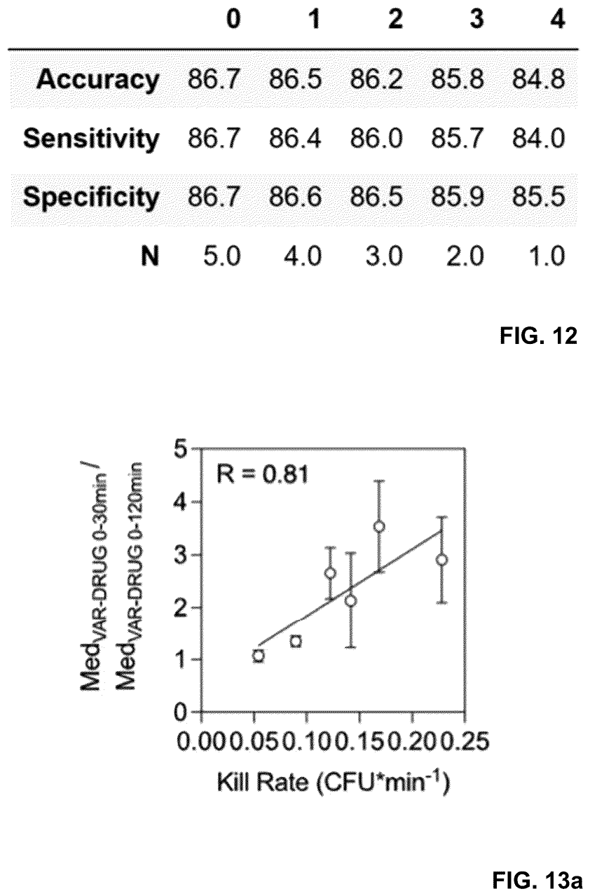

In the table depicted in FIG. 12, the performance of a feature selection algorithm being based on Pareto optimality is presented. Apart from the accuracy (percentage of correctly classified experiments), the sensitivity (percentage of correctly classified experiments with susceptible strains) and the specificity (percentage of correctly classified experiments with resistant strains) is estimated. N means the number of signal parameters used by the algorithm. In the presented example, the feature selection algorithm was a classification algorithm for cell antibiotic susceptibility estimated by 300 times repeated 3-fold stratified cross-validation method. The results were obtained for a dataset counting 1102 experiments with clinical strains of E. coli and antibiotic—ceftriaxone. 561 experiments were done with susceptible strains and 541 with resistant strains. Each column of the table corresponds to different vectors of signal parameters that become inputs of the Pareto optimal classification algorithms with maximum accuracy and minimum number of signal parameters. The accuracy ranges from 84.8% for a single signal parameter based algorithm up to 86.7% for a five signal parameters based algorithm. Addition of subsequent signal parameters improves sensitivity from 84.8% to 86.7%.

### Correlation to the Kill Rate of an Organism

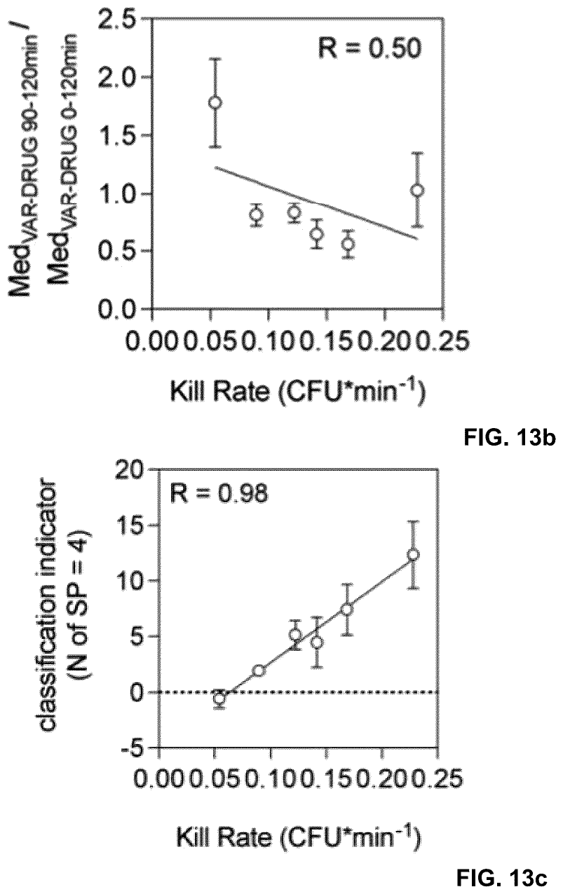

With reference to FIGS. 13a to 13c the method according to the invention is shown to correlate to the kill rate of an organism.

That is, susceptible cells exposed to a toxin or antibiotic exhibit a decrease in viability resulting eventually in death. In the case of bacteria exposed to a bactericidal antibiotic, a kill rate can be measured by determining the change of the number of colony-forming units (CFUs) at different time points. A CFU is thereby defined as a bacterium (=a unit), that through multiple replication cycles forms a visible colony on growth permitting medium. The number of these CFUs is therefore a measure of the concentration of viable bacteria in the bacterial suspension of interest. Multiple samplings over time assess the change in CFU numbers of the surviving bacteria and therefore serve to calculate a kill rate. This method has been a very reliable standard technique for more than a century already applied by for instance Robert Koch.

However, one of the major drawbacks is its dependence on growth and thus the time a bacterium needs to form a visible colony for analysis. Fast growing bacteria like K. pneumoniae form colonies visible by eye in ten hours, slow growing pathogens like M. tuberculosis however take several weeks. In any case, it is a very reliable but slow method.

In the present example depicted in FIGS. 13a to 13c, different susceptible strains of the bacterium K. pneumoniae exhibit different kill rates when exposed to the antibiotic ceftriaxone depending on a range of reasons and biological constituents. In our test group of six K. pneumoniae isolates, the kill rates ranged from 0.05 to 0.25 CFU*min⁻¹. For the determination of the kill rate, strains were cultured similarly to those described previously and were exposed to the same concentration of ceftriaxone. The population was sampled every 20 minutes and the number of viable bacteria was determined through the number of CFUs after plating for colonies and incubation over night of culture plates. At least three experiments were performed per strain. The same strains were used in parallel for detecting the deflections of the flexible support with the motion detector in the same sequence of recording phases as shown in FIGS. 7a to 7b. The same two signal parameters SP1: Median_{VAR-DRUG 0-30 min}/Median_{VAR-DRUG 0-120 min} and SP2: Median_{VAR-DRUG 90-120 min}/Median_{VAR-DRUG 0-120 min} were used separately to test the correlation between the detected signal and the kill rate. In doing so at least three signal recordings with the motion detector with complete Blank, Bac and Drug phases were used to calculate the Mean and SEM of both signal parameters derived from the Drug phase. A positive correlation with R_{SP1}=0.81 and R_{SP2}=0.5 was observed, suggesting a correlation between the deflections of the flexible support of the motion detector and the kill rate, see FIGS. 13a and 13b.

However, and as follows from FIG. 13c, the correlation significantly improved by increasing the number of signal parameters to calculate a classification indicator (in this case four signal parameters: Median_{VAR-DRUG 90-120 min}/Median_{VAR-DRUG 0-120 min}, Median_{VAR-DRUG 0-30 min}/Median_{VAR-DRUG 0-120 min}, Median_{VAR-DRUG 60-90 min}/Median_{VAR-DRUG 30-60 min}, Median_{VAR-DRUG 60-90 min}/Median_{VAR-DRUG 0-120 min}). The correlation of the kill rate (CFU*min⁻¹) and the classification indicator from the signal improved to an R=0.98. So the linear combination of more than two signal parameters in order to calculate a classification indicator helps describe the biological phenomenon of reaction to a stress environment (antibiotic susceptibility) more accurately. Hence, in summary it can be said that the signal of the motion detector reflects the kill rate of an organism, and wherein the linear combination of the signal parameters improves the correlation to a kill rate determined with standard microbiological methods. High correlation of the classification indicator means that it is able to predict the kill rate and is an example of a linear regression algorithm.

### Classification of Metabolic Activity

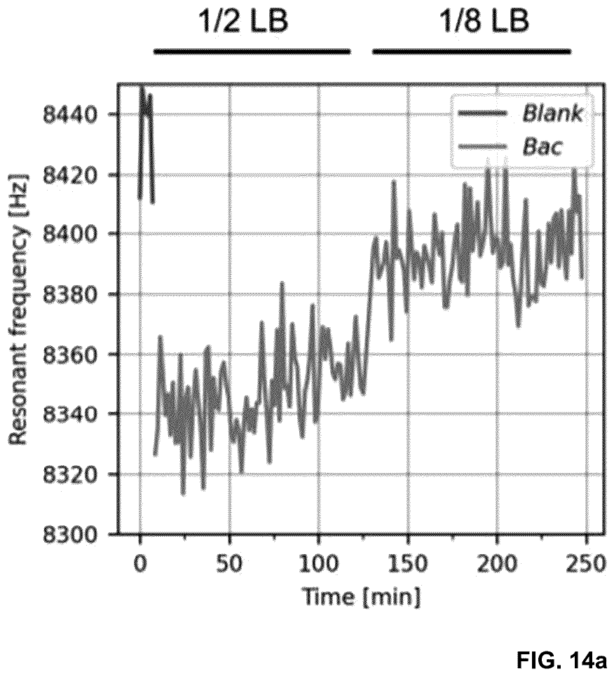

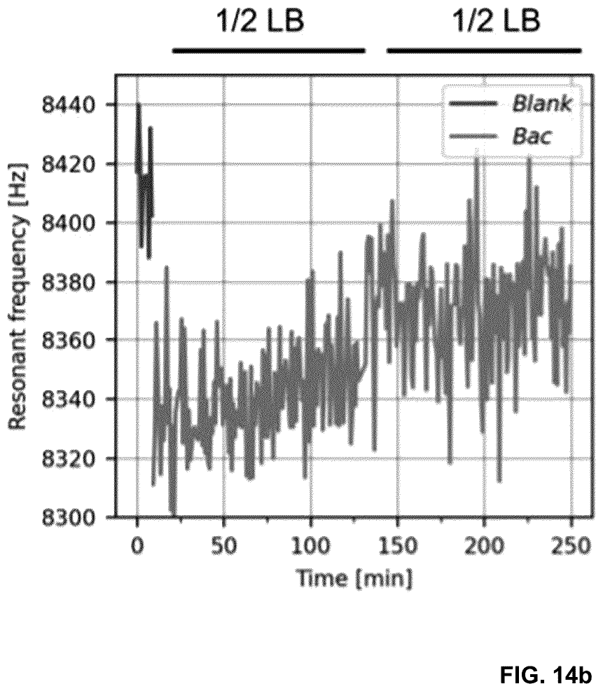

With reference to FIGS. 14a to 16b it is illustrated that the method according to the invention can be used to classify metabolic activity.

In the example depicted in FIGS. 14a and 14b, the time series of the signal estimator, the resonant frequency, is analysed and similar to the previous examples the signal parameters are extracted. FIG. 14a depicts the signal estimator for E. coli ATCC-25922 attached to a microcantilever when being exposed to ½ LB and subsequently to ⅛ LB, i.e., E. coli cells experience different nutrient availability and therefore exhibit different metabolic activity. In a control experiment depicted in FIG. 14b, the same strain is exposed to ½ LB replaced by ½ LB. When comparing FIGS. 14a and 14b it becomes apparent that the increase of the resonant frequency is less pronounced for a change from ½ LB to ½ LB (“unchanged” condition) compared to the exchange of media from ½ LB to ⅛ % LB. Therefore, in principle, signal parameters can be extracted and can be used to discriminate between metabolic states of the same organism.

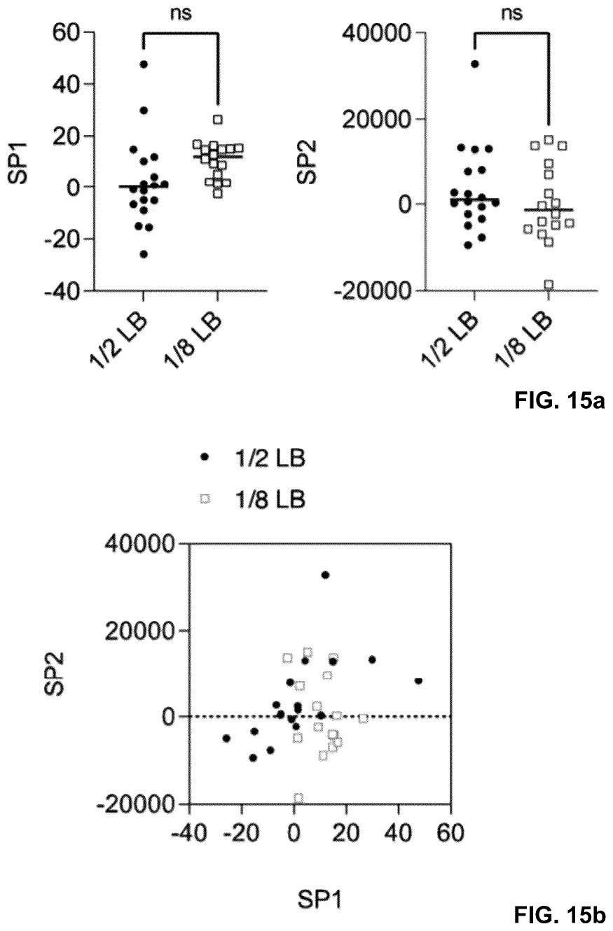

FIGS. 15a and 15b depict extracted signal parameters that were determined from the resonant frequency domain. As follows from the graphs depicted in FIG. 15a, none of them can sufficiently separate or discriminate between the two populations to an acceptable degree (t-test, p>0.05). From the graph depicted in FIG. 15b showing the combination of the two signal parameters SP 1 and SP 2, it follows that there is an overlap of the recordings of E. coli in ½ LB and E. coli in ⅛ LB.

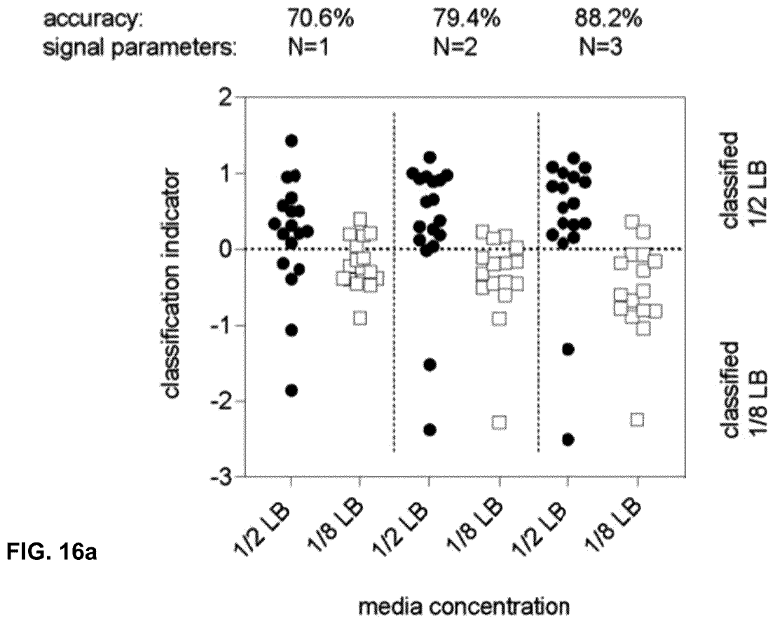

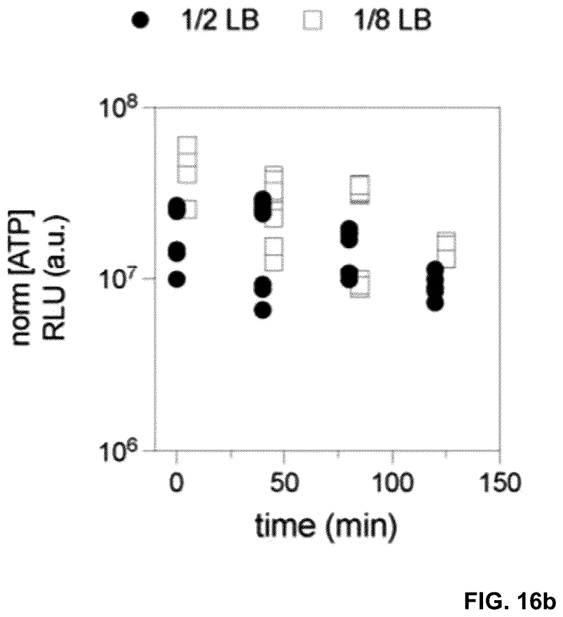

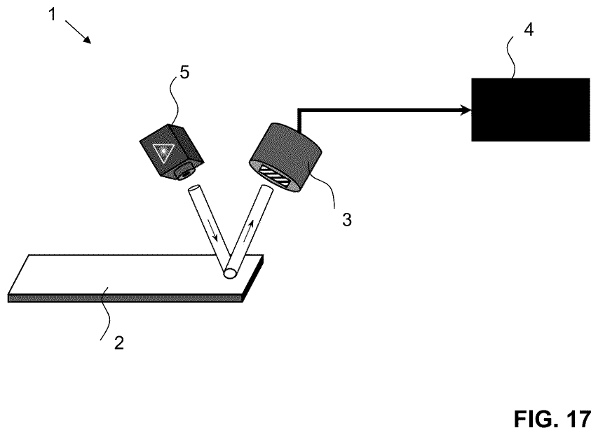

As mentioned earlier and as illustrated in FIG. 16a, line-separating the populations exposed to ½ LB and ⅛ LB can be expressed by linear combination of these two signal parameters. In fact, using a linear combination of more than two signal parameters (in this case three) can further improve the separation. This linear combination of signal parameters becomes a classification indicator. As follows from FIG. 16a, the accuracy of separating the two populations rose from 70.6% using only one signal parameter to 88.2% using three signal parameters. The weights and the threshold are estimated using a logistic regression algorithm, that is the linking algorithm is a logistic regression algorithm here.

In order to structuralize the description of signal parameters the polish notation together with some formal structure of signal parameters names are used.

The signal parameters names have the following structure

TIME_SERIES_NAME_{——}PHASE_NAME_{——}START_TIME-
END_TIME_{——}STATISTICS_USED

(e.g f0_Bac__80-90_p50—means f0 signal estimator for which 50^{th} percentile is calculated for bac phase for time interval from 80 minute to 90 minute). Statistics used are percentiles, means, standard deviation (std), and slope.

Polish notation is a way to code arithmetic expressions without using parentheses e.g.

/+a b−a b is equivalent to (a+b)/(a−b). It is used, to automatically code signal parameters being ratios and differences of other signal parameters.

The feature selection algorithm selected the following noise signal parameters as pareto optimal based on accuracy and number of signal parameters:

- f0_{——}Bac_{——}80-90_p50 f0_{——}Bac_{——}0-10_p50
- A_{——}Bac_{——}110-120_p50 A_{——}Bac_{——}0-120
/ Q_{——}Bac_{——}80-90_{——}p50 f0_{——}Bac_{——}20-30_{——}p50
/ f0_{——}Bac_110-120_p50 Q_{——}Bac_{——}70-80_p50
- f0_{——}Bac_{——}0-10_p50 f0_{——}Bac_{——}0-120_{——}p50.

FIG. 16b relates to an independent experiment using a chemiluminescent method to measure ATP concentrations. Similar to the measurement present with respect to FIG. 15a to 16a, the energy state of a cell can be assessed by the concentration of ATP in different media with different nutrient availability (½ LB and ⅛ LB) and can be assessed by using enzymatic assays using chemiluminescence produced by the ATP dependent enzymatic activity of a luciferase (BactiterGlo™).

As follows from FIG. 16b, one metabolic marker of metabolic activity of a cell likewise results in two partially overlapping populations, wherein each symbol in the graph represents one measurement. Thus, similar to the case where one signal parameter is used to analyse the signals of the flexible support in the motion detector, using only one metabolic marker is insufficient for separating two metabolic states of the same type of cells in narrowly related environments (in this case ½ LB and ⅛ LB). Thus, using a combination of signal parameters, i.e., a classification indicator, can be understood as to capture several aspects of the metabolic states of a cell in this case as well. Increasing the number of signal parameters can be understood similarly to increasing the number of metabolic markers.

The following embodiments explore three aspects of the time-dependency of the signal being evaluated by the evaluation device: the complex aspects of power spectral densities not covered by theoretical noise models, the related to the particle vibrations scarce events in the signal that might vanish during power spectral density estimation and self-affinity of cell vibrations.

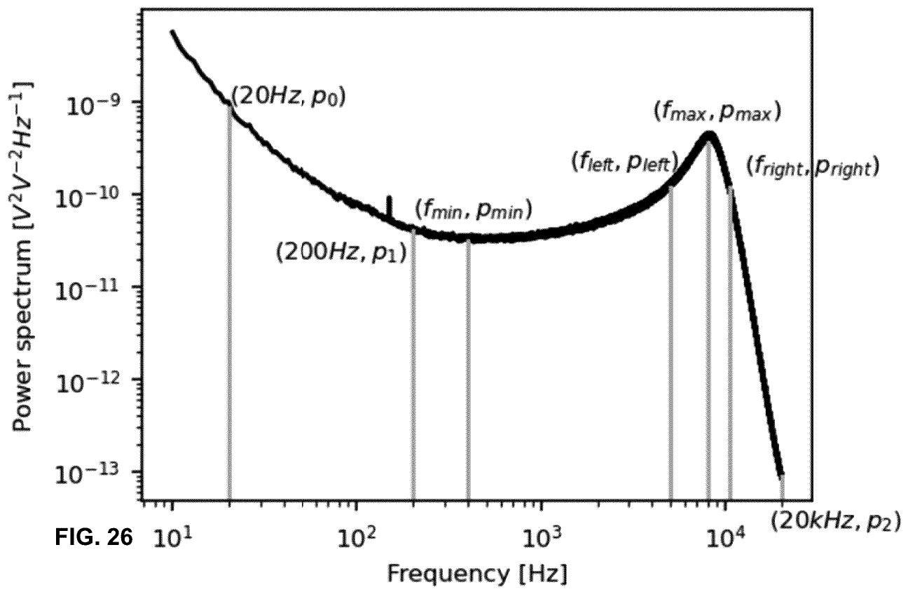

In particular, a typical spectrum and signal estimators that could be derived from a signal being detected with a motion detector and being analysed with the method according to the invention are presented in FIG. 26. For this kind of spectrum the following list of 60 time series of signal estimators are defined. They are estimated every 2 minutes.
The local minimum point of the spectrum (fₘᵢₙ and pₘᵢₙ)
The local maximum point of the spectrum (fₘₐₓ, pₘₐₓ)
The points in the middle of vertical distance between local minimum and maximum p_{left}=p_{right}=(pₘₐₓ+pₘᵢₙ)/2 and corresponding frequencies(f_{left}, f_{right})
The parameters of the flicker noise for the frequency range from 20 to 200 Hz (N0, a)

$$PSD ( f ) = \frac{{N}_{0}}{{f}^{a}} \qquad ( 4 )$$

The area under the curve for five frequency ranges (20 Hz, fₘᵢₙ), (fₘᵢₙ, f_{left}), (f_{left}, fₘₐₓ), (fₘₐₓ, f_{right}), (f_{right}, 20 kHz), from N1 to N5.
The area under the curve for 20 frequency ranges equidistant in log scale (20-28, 28-39, . . . , 224-316, 316-447, . . . , 10023-14158, 14158-20000 Hz) from NE1 to NE20.

The normalised power normNᵢ and normNEᵢ for every i Nᵢ/(N₁+N₂+N₃+N₄+N₅) and NEᵢ/(N₁+N₂+N₃+N₄+N₅) These time series of signal estimators can be used to calculate signal parameters and/or their ratios and differences.

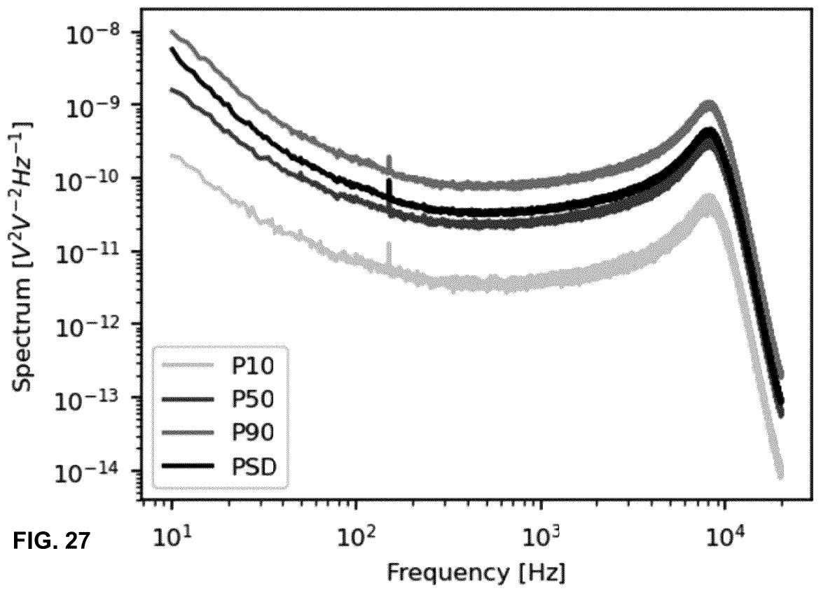

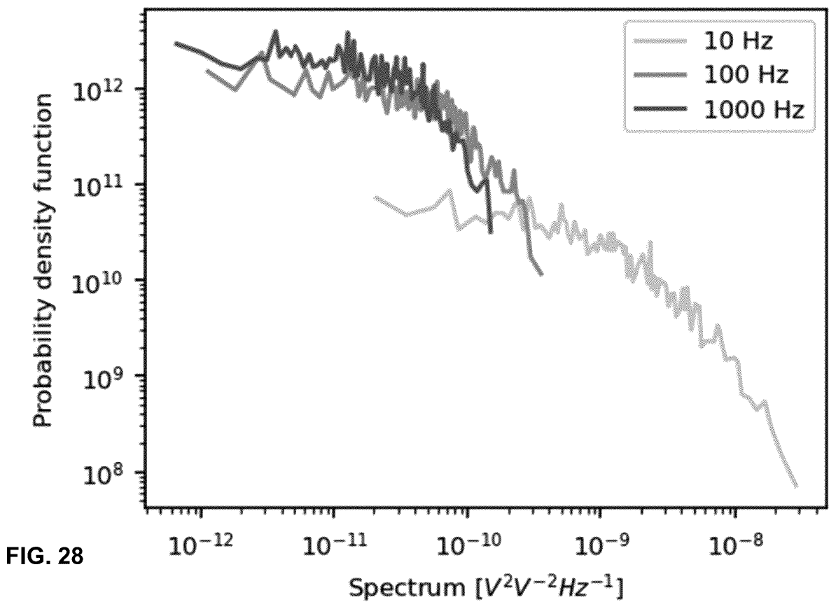

As follows from FIGS. 27 and 28, quantiles in particular percentiles of periodograms (see FIG. 27) can be used to take into account the shape of the probability distribution (see FIG. 28) of periodograms. In the described solution the 9 percentiles from 10^{th}, 20^{th}, . . . , 90^{th} are used and they are calculated for predefined frequency ranges. In the presented embodiments the following frequency ranges are used: 0-10, 10-100, 100-200, 200-400, 400-1000, 1000-2000, 2000-4000, 4000-5000, 5000-6000, 6000-7000, 7000-8000, 8000-10000 Hz (p10_1000-2000 means 10^{th} percentile for frequency range 1000-2000 Hz). In addition the following extended time series are created.

KLPQ—Kullback-Leibler divergence between Chi² probability density function and empirical probability density function calculated basing on percentiles,

KLQP—Kullback-Leibler divergence between empirical probability density function calculated basing on percentiles and Chi² probability density function,

Kurtosis—is kurtosis (a moment) of empirical probability density function,

JS—is Jensen-Shannon distance between Chi² probability density function and empirical probability density functions,

MO—(p₆₀−p₄₀)/(p₉₀−p₁₀) (ratios and differences)

EEPDF—is mean (a moment) of the empirical probability density function

They provide every 5 min samples of 112 time series of signal estimators from which signal parameters and their ratios and differences for different percentiles, frequency ranges and time intervals are calculated. They are objects of the feature selection algorithm.

The current findings about cell vibrations revealed that their power spectral density function has strong 1/f characteristic. This kind of spectrum is often connected with signal self-affinity. Self-affinity can be analysed by detrended fluctuations analysis (DFA) and its generalisation multifractal detrended fluctuations analysis (MF-DFA). In these methods the dependence of the signal statistical properties on the time scale is investigated. In the presented embodiments, the MF-DFA based signal parameters were used. In particular, the following 21 generalized mean exponents/powers are used: −10.0, −8.0, −6.0, −4.0, −2.0, −1.0, −0.8, −0.6, −0.4, −0.2, 0.0, 0.2, 0.4, 0.6, 0.8, 1.0, 2.0, 4.0, 6.0, 8.0, 10.0. Together with 9 numbers of subintervals (4, 8, 16, 32, 64, 128, 256, 512, 1024) results in 189 signal parameters, from which the ratios and or differences are further calculated.

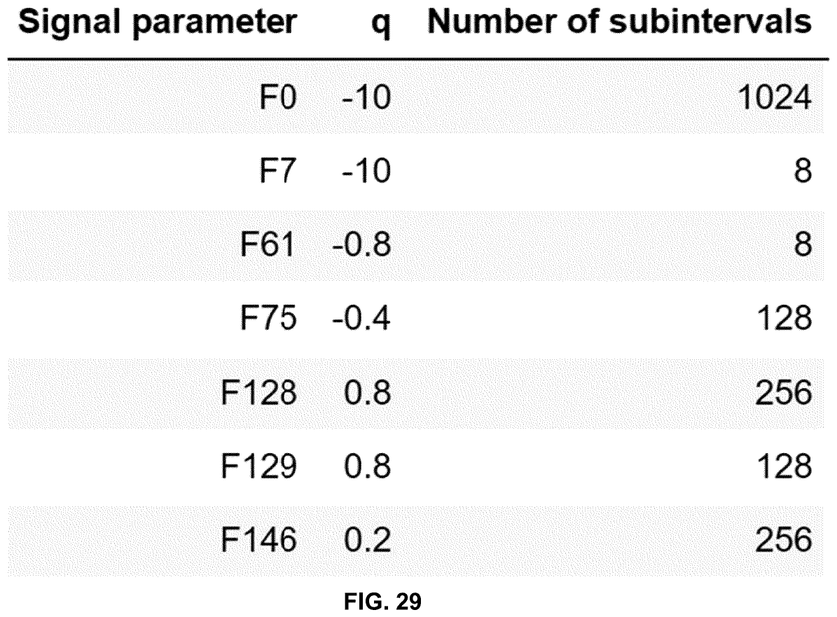

In FIG. 29 the table of the signal parameters used in the presented embodiments are shown. They are calculated as a ratio of F_{q}(number of subintervals) for the last 20 min of the drug phase and the first 20 min of the drug phase. So the signal parameter “F0” is F₋₁₀(1024) of the last 20 minutes of the drug phase divided by F₋₁₀(1024) of the first 20 minutes of the drug phase. These signal parameters are again mixed by calculation of their ratios (e.g. F146_F129 means F146/F129).

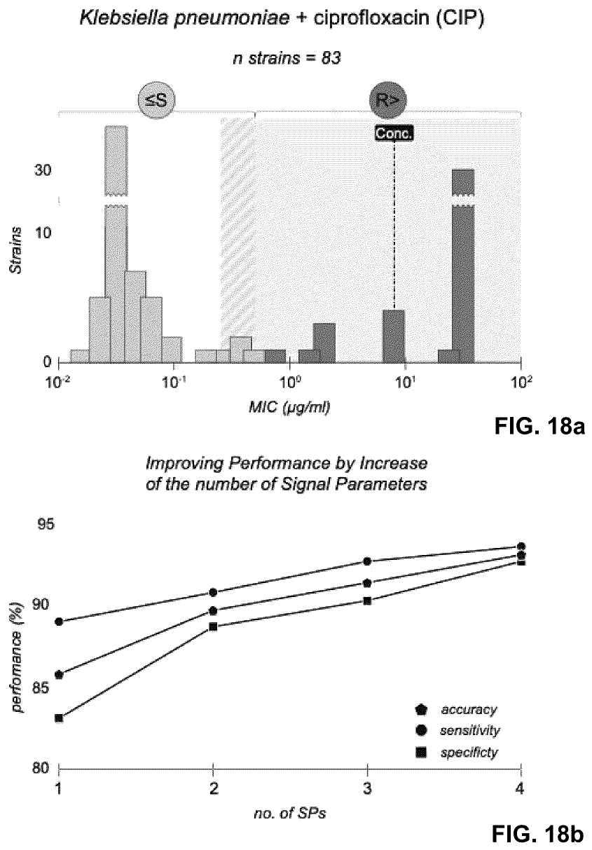

Analogous to the examples consisting of relatively small datasets depicted in FIGS. 7a to 12 and 14 to 16a, the combination of signal parameters (SPs) substantially increases the performance of a linking algorithm being a classification algorithm on big data sets comprising exceedingly diverse specimens. Again, we used for a classification the reaction of cells changing from a favourable culture environment into a stress environment, this time with the antibiotic ciprofloxacin (CIP). We used 83 different clinical isolates of Klebsiella pneumoniae strains whose minimal inhibitory concentrations (MICs) to ciprofloxacin differ and range over four orders of magnitude, being either considered clinically susceptible or resistant, depicted in FIG. 18a as <S and >R, respectively. MICs were determined by standard diagnostic reference methods (E-test or broth micro dilution). The level of resistance to ciprofloxacin is due to different resistance mechanisms and other genetic determinants of the different strains. Despite the mentioned strain diversity, the nanomotions of the 83 strains were measured at only one concentration, 8 μg/ml CIP (“Conc.” in FIG. 18a) in 233 experiments, in an experimental setup of nanomotion recordings previously described for FIGS. 7a and 7b. In brief, in each experiment the bacterial specimen attached to the micromechanical sensor was exposed to culture media (½ concentrated LB) for 120 min followed by 120 min exposure to CIP.

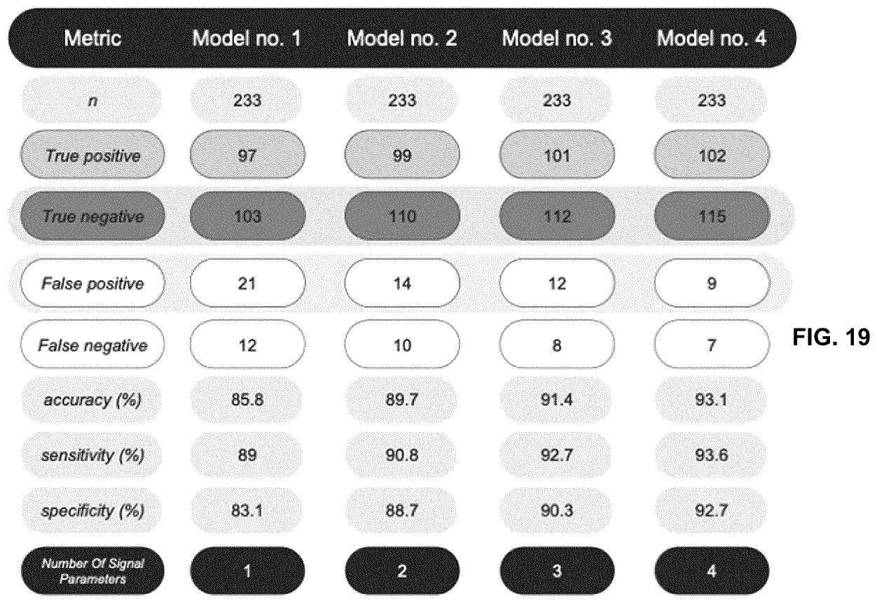

In FIG. 18b the improving performance depending on the increasing number of SPs is visualised and FIG. 19 lists all metrics assessed for the linking algorithm being a classification algorithm of this dataset. Accuracy, sensitivity and specificity are calculated as follows:

$$Accuracy ( \% ) = 100 \frac{true positives + true negatives}{true positives + true negatives + false positives + false negatives}$$

$$Sensitivity ( \% ) = 100 \frac{true positives}{true positives + false negatives}$$

$$Specificity ( \% ) = 100 \frac{true negatives}{true negatives + false positive}$$

In this, True positives are considered correctly classified experiments with susceptible strains, True negatives are correctly classified experiments with resistant strains, False positives are experiments with falsely classified resistant strains, and False negatives refer to experiments with falsely classified susceptible strains.

Classification model no. 1 (classification model is an instance of a linking algorithm being a classification algorithm) is based on a single SP (F128) with arguably the biggest impact, leading to a classification accuracy of 85.8%. Model no. 2 with two SPs (F128, F61) achieved 89.7% accuracy, with three SPs (F128, F61, F146_F129) 91.4%, and finally, 93.1% accuracy was reached with model no. 4 combining four SPs (F128, F61, F146_F129, F7_F0)—summarised in FIG. 19. FIG. 18b also shows the increase in sensitivity and specificity.

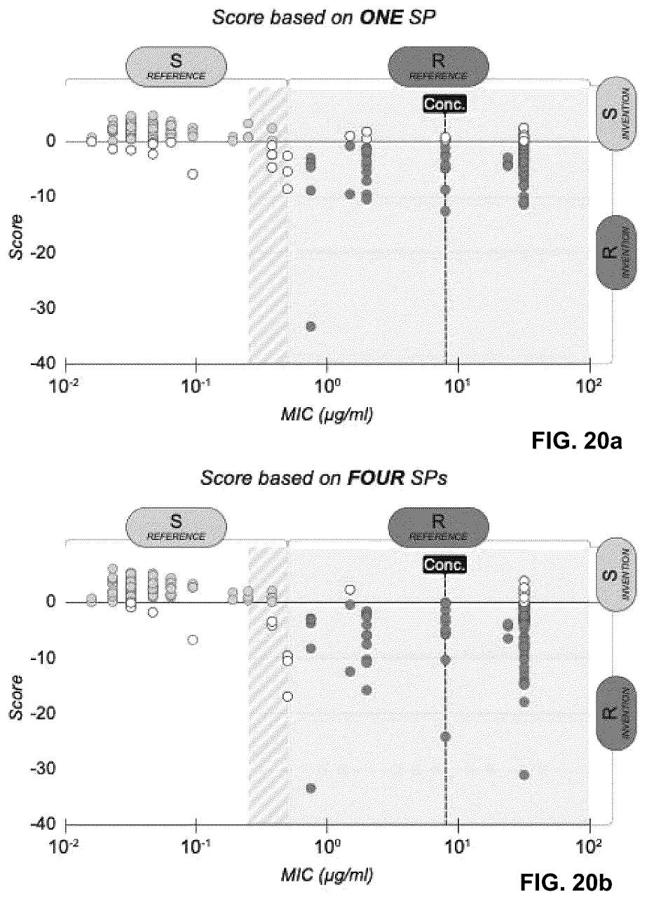

The score, i.e. the classification indicator for each of the 233 experiments is benchmarked against the reference MICs that are either considered susceptible (S REFERENCE) or resistant (R REFERENCE) in FIG. 20a, for model no.1 based on one SP, and FIG. 20b, for model no. 4 based on four SPs. The score assumes positive values for predicted susceptibility (S INVENTION) and negative values for predicted resistance (R INVENTION). Correctly classified experiments are depicted in light grey for susceptible strains, True positives, and dark grey for resistant strains, True negatives. The falsely classified experiments, False positives and False negatives, are shown by white circles. The number of falsely classified experiments drops from initially 33 for model no. 1 to 16 for model no. 4. All models are linking algorithms being classification algorithms being logistic regression algorithm using MF-DFA signal parameters.

In conclusion, one SP, that can be considered a single aspect of the nanomotion signal, is less suited than 4 SPs for describing the diverse response to the drug found in such a diverse dataset of 83 different strains. The increase of SPs and their combination significantly improves the performance of the classification algorithm.

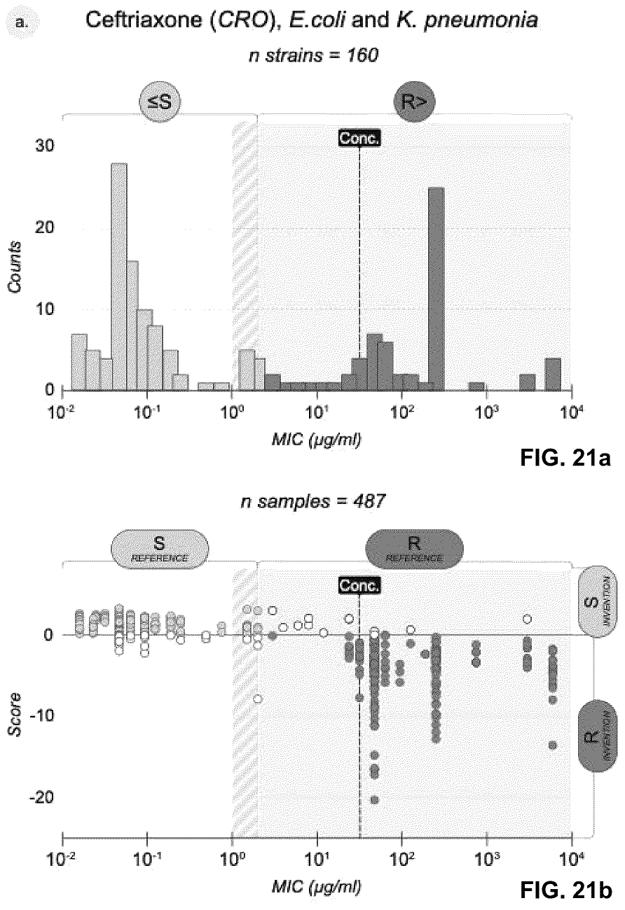

In another example, an even bigger and more diverse dataset of 487 samples comprising 160 clinical isolates of two different bacterial species, E. coli and K. pneumoniae, were exposed to one concentration of ceftriaxone (indicated as “Conc.”, FIG. 21a). The nanomotions of the bacterial cells were measured in the same experimental setting described for 7a and 7b of a 120 min media phase (½ concentrated LB) followed by a 120 min drug exposure phase (ceftriaxone, CRO), and the 160 strains again spanned a wide range of several orders of magnitude of MICs (FIG. 21a, using the standard reference method for MIC). The signal parameters being derived from signal estimators being geometrical properties of a power spectral density function are used. The feature selection algorithm selected the following signal parameters basing on accuracy, number of signal parameters and pareto optimality concept:

/ NE0_{——}Drug_{——}90-120_p50 NE0_{——}Drug_{——}0-30_{——}p50
/ normNE19_{——}Drug_{——}90-120_{——}p50
normNE19_{——}Drug_{——}60-90_{——}p50
/ normNE13_{——}Bac_{——}90-120_{——}p50
normNE13_{——}Bac_{——}90-120_{——}p50
/ normNE1_{——}Drug_{——}90-120_{——}p50 normNE1_{——}Drug_{——}0-120_{——}p50

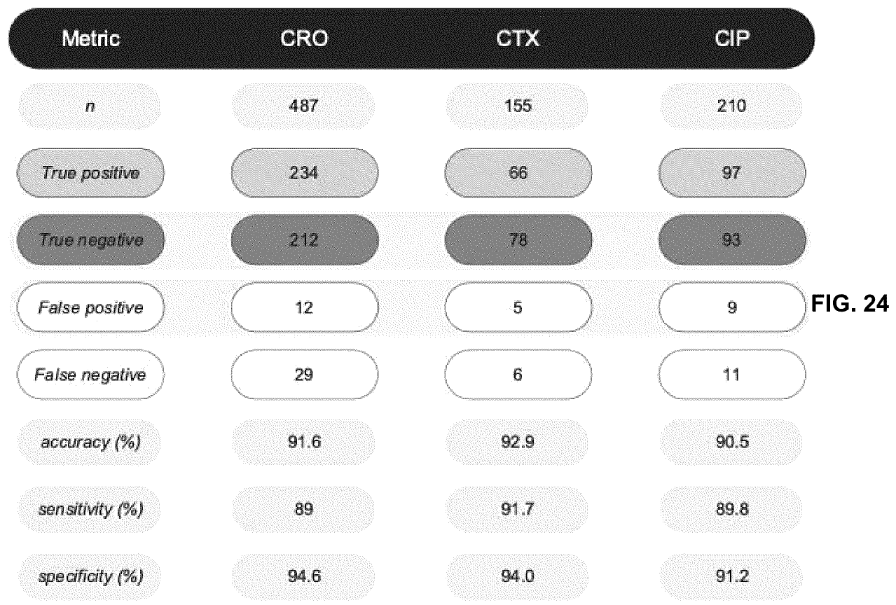

The linking algorithm being classification algorithm with a high performance of separating experiments with resistant strains from experiments with susceptible strains is based on the combination of the four signal parameters described above resulting in a score, i.e., classification indicator that assumes positive values for experiments with predicted susceptibility (S INVENTION) and negative values with experiments of predicted resistance (R INVENTION). The accuracy reached 91.6%, sensitivity 89.0% and the specificity 94.6% (FIG. 24). The scores of each sample were plotted against the reference MIC according to the method shown in FIG. 21b. Correctly classified experiments are depicted in light grey for susceptible strains (True positives), dark grey for resistant strains (True negatives). The falsely classified experiments (False positives and False negatives) are shown by white circles.

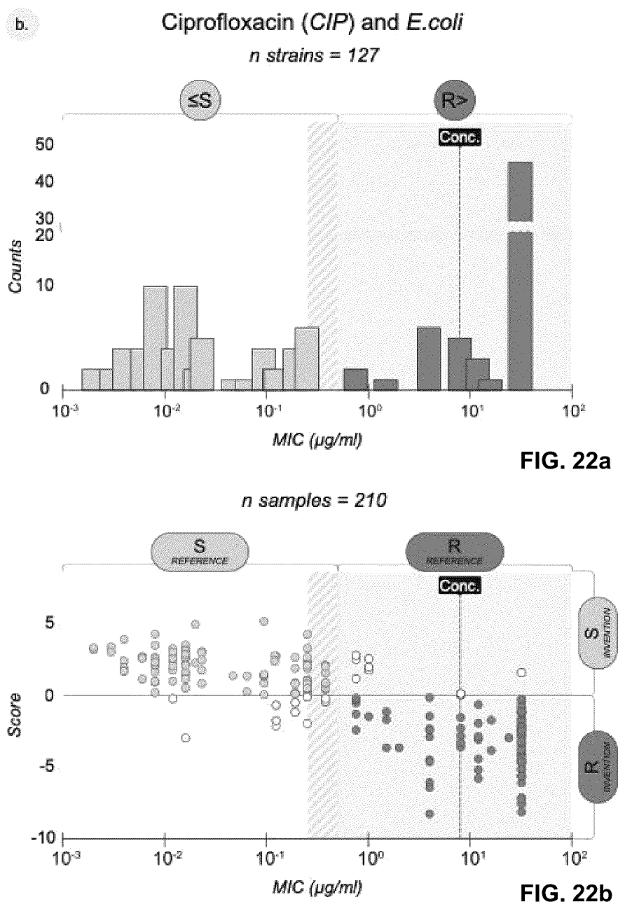

A similar analysis of 210 samples of 127 E. coli strains exposed to 8 μg/ml ciprofloxacin (CIP, indicated as “Conc.” in FIGS. 22a and 22b) was measured in the nanomotion setup explained for FIGS. 7a and 7b. After processing of the nanomotion signal, the quantile and MF-DFA signal parameters are used in this embodiment together with linking algorithm being classification algorithm being logistic regression. The signal parameters selected by the feature selection algorithm are as follows:

/ / / - p90_0-10_{——}Drug_{——}90-120_{——}mean p50_0-10_{——}Drug_{——}90-120_{——}mean - p50_0-
10_{——}Drug_{——}90-120_{——}mean p10_0-10_{——}Drug_{——}90-120_{——}mean / - p90_5000-
6000_{——}Drug_{——}90-120_{——}mean p50_5000-6000_{——}Drug_{——}90-120_{——}mean - p50_5000-
6000_{——}Drug_{——}90-120_{——}mean p10_5000-6000_{——}Drug_{——}90-120_{——}mean / / - p90_0-
10_{——}Drug_{——}0-30_{——}mean p50_0-10_{——}Drug_{——}0-30_{——}mean - p50_0-10_{——}Drug_{——}0-
30_{——}mean p10_0-10_{——}Drug_{——}0-30_{——}mean / - p90_5000-6000_{——}Drug_{——}0-30_{——}mean
p50_5000-6000_{——}Drug_{——}0-30_{——}mean - p50_5000-6000_{——}Drug_{——}0-30_{——}mean p10_5000-
6000_{——}Drug_{——}0-30_{——}mean
/ / p20_200-400_{——}Drug_{——}90-120_{——}mean p20_10-100_{——}Drug_{——}90-120_{——}mean / p20_200-
400_{——}Drug_{——}30-60_{——}mean p20_10-100_{——}Drug_{——}30-60_{——}mean
/ p30_10-100_{——}Bac_{——}60-90_mean p30_0-10_{——}Bac_{——}60-90_mean
F75

The final linking algorithm being classification algorithm was based on four SPs and from the 210 samples it correctly classified 190. Thus, it achieved an accuracy of 90.5%, a sensitivity of 89.8% and a specificity of 91.2% (FIG. 24). The majority of the twenty falsely classified samples (shown as white circles in FIG. 22b) were found around the clinical breakpoint that indicates the border between susceptible (<S) and resistant (R>) strains defined by the reference AST method.

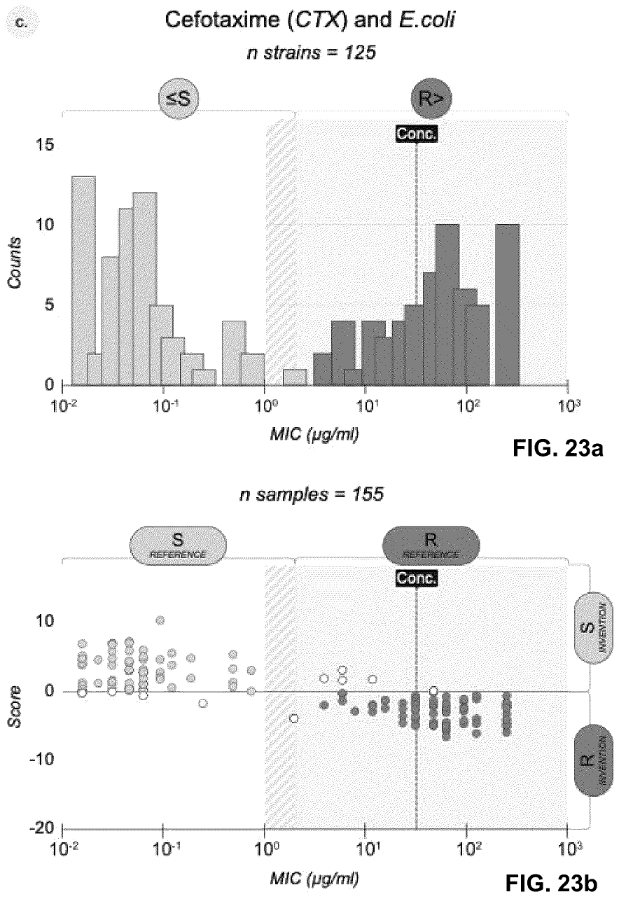

Yet, in another analysis, 155 samples of 125 diverse E. coli strains from different strain collections were exposed to a third antibiotic cefotaxime (CTX). In the same nanomotion measurement setup as described in 7a and 7b, we used 32 μg/ml cefotaxime (indicated as “Conc.” in FIGS. 23a and 23b). The quantile and MF-DFA signal parameters and linking algorithm being classification algorithm being logistic regression are used in this embodiment. The signal parameters selected by the feature selection algorithm basing on accuracy and number of signal parameters and basing on pareto optimality concept are as follows:

/ / p50_400-1000_{——}Drug_{——}90-120_{——}mean p50_100-200_{——}Drug_{——}90-120_{——}mean /
p50_400-1000_{——}Drug_{——}0-30_{——}mean p50_100-200_{——}Drug_{——}0-30_{——}mean
/ / / - p50_200-400_{——}Drug_{——}30-60_{——}mean p10_200-400_{——}Drug_{——}30-60_{——}mean -
p90_200-400_{——}Drug_{——}30-60_{——}mean p50_200-400_{——}Drug_{——}30-60_{——}mean / - p50_100-
200_{——}Drug_{——}30-60_{——}mean p10_100-200_{——}Drug_{——}30-60_{——}mean - p90_100-
200_{——}Drug_{——}30-60_{——}mean p50_100-200_{——}Drug_{——}30-60_{——}mean / / - p50_200-
400_{——}Drug_{——}0-30_{——}mean p10_200-400_{——}Drug_{——}0-30_{——}mean - p90_200-400_{——}Drug_0-
30_{——}mean p50_200-400_{——}Drug_{——}0-30_{——}mean / - p50_100-200_{——}Drug_{——}0-30_{——}mean
p10_100-200_{——}Drug_{——}0-30_{——}mean - p90_100-200_{——}Drug_{——}0-30_{——}mean p50_100-
200_{——}Drug_{——}0-30_{——}mean
F186_F64
F45_F38
/ / p70_1000-2000_{——}Drug_{——}90-120_{——}mean p70_100-200_{——}Drug_{——}90-120_{——}mean /
p70_1000-2000_{——}Drug_{——}00-30_{——}mean p70_100-200_{——}Drug_{——}0-30_{——}mean

The linking algorithm being classification algorithm based on five SPs led to only 11 false classifications, resulting in an accuracy of 92.9%, a sensitivity of 91.7% and a specificity of 94% (FIG. 24). Each sample's score, i.e., the classification indicator, is again benchmarked against the reference AST method in FIG. 22b.

In summary, for each of the four bacteria-drug combinations in FIG. 18a to 24 different highly performing linking algorithms being classification algorithms were developed based on a small number of SPs calculated from the time series of signal estimators. In each case, the group of resistant cells could be almost perfectly separated from susceptible cells, arguably based on the different cellular and metabolic responses to the three different drugs, ceftriaxone, ciprofloxacin and cefotaxime within 120 min of exposure.

The two aforementioned drugs cefotaxime and ciprofloxacin impede different cellular processes of the bacterial cell. Cefotaxime belongs to the family of beta-lactam antibiotics interfering with the cell wall metabolism while ciprofloxacin as a quinolone binds topoisomerases involved in DNA folding, a process that impact replication and effectively all processes in which the DNA is involved. The proper functioning of both drug targets is essential. While for both, their impediment is in the long run detrimental for the cell, both their triggered cellular stress responses differ.

On a total dataset size of 404 experiments, of which 202 were performed with susceptible E. coli strains and 32 μg/ml cefotaxime and an equal number of experiments with susceptible E. coli strains and 8 μg/ml ciprofloxacin, the combination of nanomotion recordings and machine learning was applied to develop linking algorithms being classification algorithms to separate the information entailed in the nanomotion response to cefotaxime from ciprofloxacin. All 404 experiments were used simultaneously to develop the classification model. If an experiment was performed with cefotaxime and afterwards correctly predicted as such by the method, it was considered correctly classified. The same accounted for ciprofloxacin. The experimental setup was again identical to the one described for FIGS. 7a and 7b with 120 min medium phase followed by 120 min exposure to either of the two drugs. For benchmarking, the MIC was determined by reference methods.

Quantile signal parameters are used and linking algorithm being classification algorithm being a support vector machine algorithm with radial basis functions. The feature selection algorithm selected the following pareto optimal signal parameters on the basis of accuracy and number of signal parameters:

- KLQP_200-400_{——}Drug_{——}90-120_{——}p95 KLQP_200-400_{——}Drug_{——}0-30_{——}p95
/ EEPDF_0-10_{——}Drug_{——}090-120_{——}p95 EEPDF_0-10_{——}Drug_{——}0-30_{——}p95
/ Kurtosis_0-10_{——}Drug_{——}90-120_{——}p10 JS_0-10_{——}Drug_{——}90-120_{——}p10
- EEPDF_{——}Drug_{——}90-120_{——}mean JS_2000-4000_{——}Drug_{——}0-30_{——}mean
MO_0-10_{——}Drug_{——}90-120_{——}p75
MO_100-200_{——}Drug_{——}30-60_{——}std
KLQP_0-10_{——}Drug_{——}0-30_{——}std

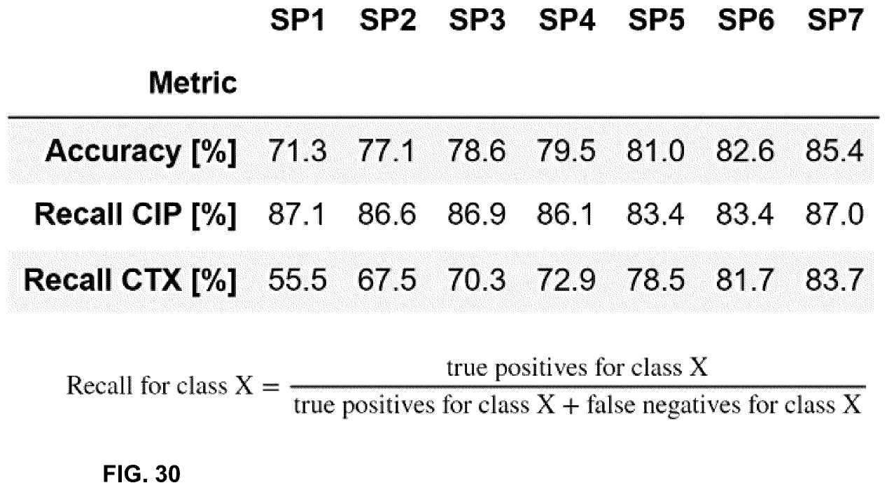

In a linking algorithm being classification algorithm the predicted susceptible response to ciprofloxacin was assigned negative score values, while the predicted response to cefotaxime was assigned positive score values (similar to a classification algorithm for R and S phenotypes). Wrongly classified experiments assumed positive values for ciprofloxacin and negative values for cefotaxime, accordingly. On a high-performing classification algorithm based on seven SPs the accuracy for both drugs reached 85.4%. 87.0% of the ciprofloxacin experiments were correctly classified and 83.7% for cefotaxime. The impact of every additional signal parameter is presented in FIG. 30. Thus, the information entailed in the nanomotion signal caused by different stressors can be extracted and described by a classification algorithm.

Besides chemical stressors, as shown for different drugs (FIG. 7a to 12 and FIGS. 18a to 24) and different levels of nutrient supply (FIGS. 14 to 16a), a physical stressor can also affect the cellular and metabolic activity and thus the nanomotions of cells. To that end, nanomotions of several different strains of E. coli differing in their genetic composition were recorded in culture media (% concentrated LB) for 30 min. Half of these experiments were performed at room temperature while the other half was exposed to 37° C. (for 37° C. 76 experiments, for RT 72 experiments). The metabolic rate at 37° C. was expected to be higher as the bacterium E. coli is adapted to a lifestyle within the human gut microbiome. The spectral signal parameters and logistic regression classification algorithm are used. The feature selection algorithm has selected the following pareto optimal signal parameters basing on accuracy and number of signal parameters:

/ NE17_{——}Bac_{——}0-15_{——}p25 NE12_{——}Bac_{——}0-15_{——}p25
/ NE17_{——}Bac_{——}15-30_{——}p10 NE13_{——}Bac_{——}15-30_{——}p10
/ NE16_{——}Bac_{——}15-30_{——}p75 NE15_{——}Bac_{——}15-30_{——}p75
- NE19_{——}Bac_{——}15-30_{——}p10 NE19_{——}Bac_{——}0-15_{——}p10
N1_{——}Bac_{——}0-15_{——}std

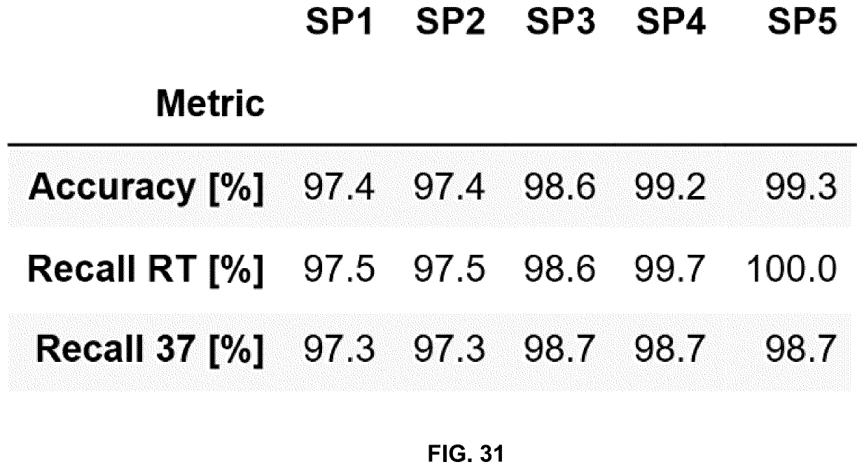

Comparing the nanomotions at two different temperatures and thus expectedly a rather global change in the cell's metabolic activity, the classification algorithm's accuracy in separating both conditions ranges from 97% with a single SP up to 99% for five SP. In FIG. 31 the dependence of algorithm performance on the number of signal parameters selected on the basis of accuracy and the number of signal parameters and Pareto optimality concept is presented.

### Drug Sensitivity Testing on Cancer Cells

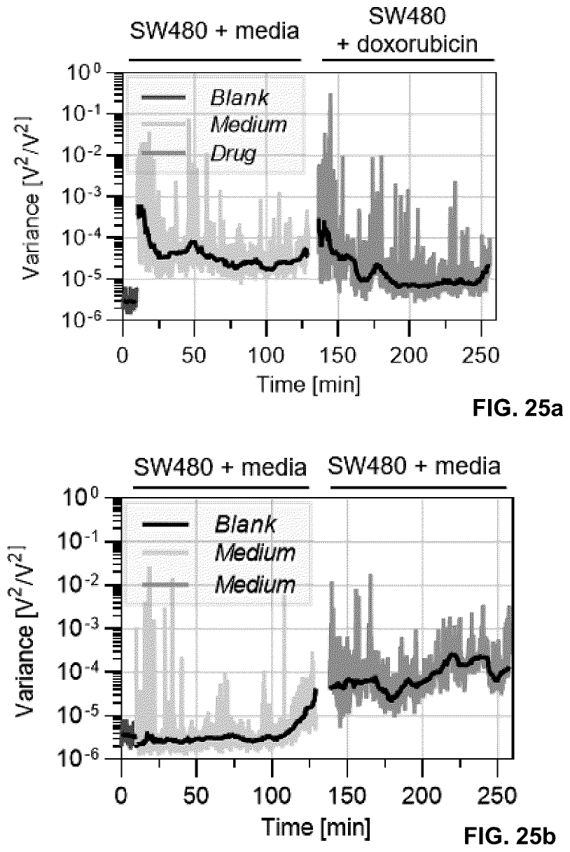

FIGS. 25a and 25b illustrate that the method according to the invention can be used to perform drug sensitivity testing (DST) on cancer cells attached to the microcantilever. In particular, these figures show the variance of the deflection of a microcantilever with attached human colon cancer cell line SW480 measured in cell culture medium and subsequent exposure to inhibitory concentrations of the drug doxorubicin (FIG. 25a) and the variance for cells kept in cell culture medium (FIG. 25b).

The doxorubicin susceptible SW480 was cultured under standard laboratory conditions (i.e., cell culture medium (Dulbecco's Modified Eagle Medium (DMEM) containing 10% heat-inactivated fetal calf serum (FCS), 37° C., 5% CO₂) in cell culture flasks. In preparation of nanomotion experiments, cells were detached, collected in cell culture medium, and washed in DMEM containing 10% FCS. The cell suspension was used for the attachment to the cantilever, and all nanomotion measurements with the device were performed in DMEM containing 10% FCS. The colon cancer cells were attached to the cantilever. Three phases of signal recording were performed: (i) Blank phase, where the deflections of the bare cantilevers are measured, (ii) the Medium phase, where the deflections of the cantilever with attached SW480 were measured followed by (iii) the Drug phase, where the deflections of the cantilever with attached SW480 after exposure to a drug were recorded or a second Medium phase without doxorubicin. The recordings presented in the following were conducted in a motion detector installed in a CO₂ supplied incubator to allow optimal culture conditions for cancer cells.

FIG. 25a depicts the variance of the nanomotion signal over time with attached SW480 colon cancer cells in culture conditions and subsequent supplementation of the medium with 32 μM doxorubicin in the Drug phase. In the experiment depicted in FIG. 25b without the addition of doxorubicin, the cells are kept in DMEM and a second Medium phase is recorded. In comparing FIGS. 25a and 25b, the decrease in the variance after exposure to the drug is prominent compared to the unchanged conditions. Based on 61 experiments with SW480 (32 with exposure to doxorubicin and 29 without doxorubicin), spectral signal parameters were calculated and linking algorithm being logistic regression classification algorithms are used for the discrimination of both conditions.

The signal parameters selected by the feature selection algorithm basing on pareto optimality, accuracy and number of signal parameters are as follows:

/ NE8_{——}Drug_{——}0-30_{——}std NE2_{——}Drug_{——}0-30_{——}std
- NE0_{——}Drug_{——}90-120_{——}p75 NE0_{——}Drug_{——}0-30_{——}p75
/ NE10_{——}Drug_{——}90-120_{——}slope NE3_{——}Drug_{——}90-120_{——}slope
- NE19_{——}Drug_{——}90-120_{——}std NE13_{——}Drug_{——}30-60_{——}std
- NE9_{——}Drug_{——}60-90_{——}std NE5_{——}Drug_0-30_{——}std
- NE7_{——}Drug_{——}0-30_{——}std NE1_{——}Drug_{——}0-30_{——}std
NE4_{——}Drug_{——}0-30_{——}p95

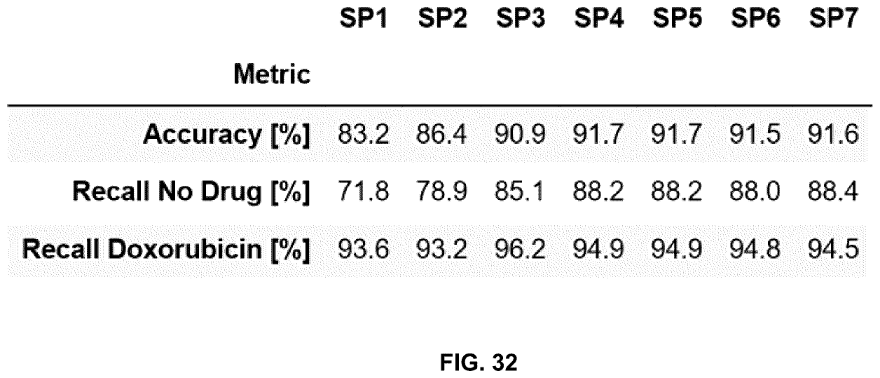

Indeed, the accuracy of pareto optimal linking algorithms ranges from 83.2% for one SP up to 91.6% for seven SPs. In FIG. 32, the dependence of algorithm performance on the number of signal parameters selected on the basis of accuracy, number of signal parameters and Pareto optimality concept is presented. The method is a forward selection method based on 300 times repeated 3-fold cross-validation.

### Quantification of Metabolic Activity

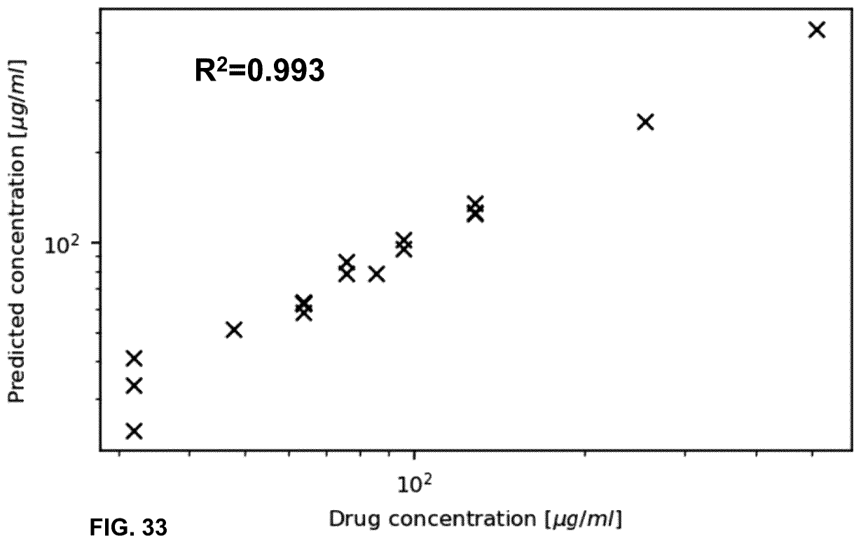

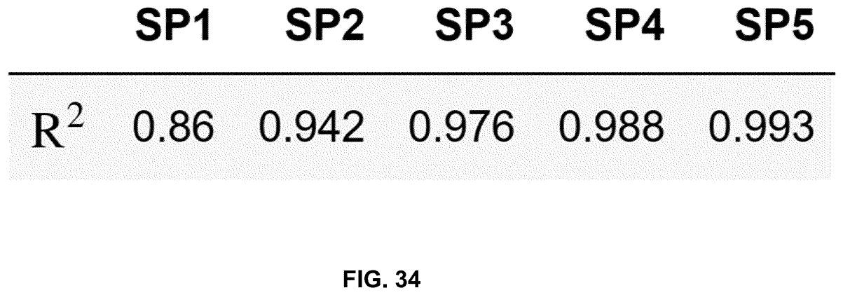

Besides qualitative assessments of metabolic states of a cell by using classification models, the impact of interference at a cell's metabolic activity by a drug can be quantified using regression models. We used the clinical E. coli isolate IMHA-2155385, which is susceptible to the drug combination ceftazidime-avibactam. This cephalosporin/beta-lactamase inhibitor combination attacks bacterial cell wall synthesis and simultaneously blocks the beta-lactamase-mediated resistance mechanism. In FIG. 33, the results of the regression of the metabolic state triggered by various inhibitory concentrations of the antibiotic is presented (32 μg/ml to 512 μg/ml). The plot shows the relation between the result of the prediction of drug concentration by five signal parameters-based models and the actual drug concentration. The coefficient of determination for the presented models ranges from 0.86 for the single signal parameter up to 0.993 for five signal parameters. In FIG. 34 the dependence of algorithm performance on the number of signal parameters selected on the basis of mean squared error and, number of signal parameters and Pareto optimality concept is presented. The method is a forward selection method based on 10-fold cross-validation. The linking algorithm being regression algorithm being linear regression algorithm is used with the following selected signal parameters:

/ / - p50_8000-10000_{——}Drug_{——}90-120_{——}mean p10_8000-10000_{——}Drug_{——}90-120_{——}mean -
p50_1000-2000_{——}Drug_{——}90-120_{——}mean p10_1000-2000_{——}Drug_{——}90-120_{——}mean / -
p50_8000-10000_{——}Drug_{——}0-30_{——}mean p10_8000-10000_{——}Drug_{——}0-30_{——}mean -
p50_1000-2000_{——}Drug_{——}0-30_{——}mean p10_1000-2000_{——}Drug_{——}0-30_{——}mean
/ / p10_6000-7000_{——}Bac_{——}90-120_{——}mean p10_0-10_{——}Bac_{——}90-120_mean / p10_6000-
7000_{——}Bac_{——}60-90_mean p10_0-10_{——}Bac_{——}60-90_mean
/ / p20_6000-7000_{——}Drug_{——}90-120_{——}mean p20_5000-6000_{——}Drug_{——}90-120_{——}mean /
p20_6000-7000_{——}Drug_{——}60-90_{——}mean p20_5000-6000_{——}Drug_{——}60-90_{——}mean
/ / / - p90_7000-8000_{——}Drug_{——}90-120_{——}mean p10_7000-8000_{——}Drug_{——}90-120_{——}mean -
p60_7000-8000_{——}Drug_{——}90-120_{——}mean p40_7000-8000_{——}Drug_{——}90-120_{——}mean / -
p90_2000-4000_{——}Drug_{——}90-120_{——}mean p10_2000-4000_{——}Drug_{——}90-120_{——}mean -
p60_2000-4000_{——}Drug_{——}90-120_{——}mean p40_2000-4000_{——}Drug_{——}90-120_{——}mean / / -
p90_7000-8000_{——}Drug_{——}0-30_{——}mean p10_7000-8000_{——}Drug_{——}0-30_{——}mean - p60_7000-
8000_{——}Drug_{——}0-30_{——}mean p40_7000-8000_{——}Drug_{——}0-30_{——}mean / - p90_2000-
4000_{——}Drug_{——}0-30_{——}mean p10_2000-4000_{——}Drug_{——}0-30_{——}mean - p60_2000-
4000_{——}Drug_{——}0-30_{——}mean p40_2000-4000_{——}Drug_{——}00-30_{——}mean
/ / p30_6000-7000_{——}Drug_{——}60-90_{——}mean p30_5000-6000_{——}Drug_{——}60-90_{——}mean /
p30_6000-7000_{——}Bac_{——}60-90_{——}mean p30_5000-6000_{——}Bac_{——}60-90_{——}mean

The presented results confirm that the invention allows measuring the increased impact of metabolic activity related to increasing drug concentration.

LIST OF REFERENCE SIGNS

| | | | |
|---|---|---|---|
| 1 | motion detector | 4 | evaluation device |
| 2 | flexible support | 5 | source of radiation |
| 3 | detection device | | |

1. A method of analysing a motional activity of particles with a motion detector, wherein the motion detector comprises:
a flexible support being configured to deflect,
a detection device for detecting signals being generated upon the deflection of the flexible support, and
an evaluation device for evaluating the detected signals,
wherein the method comprises the steps of:
bringing at least one particle into contact with the flexible support,
detecting at least one time-dependent signal that is indicative of deflections of the flexible support due to motions of the at least one particle, using the detection device, and
evaluating the detected time-dependent signal with the evaluation device,
wherein the evaluation of the detected time-dependent signal comprises:
analysing a time-dependency of the signal to derive a plurality of signal parameters from the signal that characterise a variation of the signal as a function of time, and
executing a linking algorithm that has, as input variables, an input vector comprising at least a selection of said signal parameters, and has, as an output variable, at least one activity indicator that is indicative of a motional activity of the particle.

2. The method according to claim 1, wherein, in the step of analysing the time-dependency of the signal, a plurality of time intervals are defined, wherein the time-dependency of the signal is analysed within each time interval separately in order to obtain a value for each of a plurality of signal estimators for each time interval, and wherein a time series of each signal estimator is analysed in order to obtain each of the signal parameters.

3. The method according to claim 2, wherein at least one of:
i) each signal estimator value is determined by at least one of: fitting a noise model to the signal or by applying at least one statistical algorithm to the signal, or
ii) each signal estimator is selected from the group consisting of estimators of moments of probability density functions, geometrical properties of power spectral density functions, percentiles of probability density functions, correlation parameters, partial correlation parameters, parameters of auto-regressive moving average models and parameters of nonlinear auto-regressive moving average models.

4. The method according to claim 2, wherein each signal estimator is a geometrical property of a power spectral density function.

5. The method according to claim 2,
Determine, for each time interval, a plurality of periodograms, and
For all periodograms, determine at least one quantile for at least one frequency range, whereby the signal estimator is obtained.

6. The method of claim 5, wherein the time series of at least one of the signal estimators is extended so as to form an extended time series by at least one of:
Combining at least two signal estimators into at least one of differences or ratios thereof;
Transforming a plurality of quantiles into values of an empirical probability density function,
and
wherein the at least one signal parameter is obtained from the extended time series.

7. The method according to claim 2, wherein at least one signal parameter is derived from at least one of: the time series of at least one of the signal estimators and/or from the extended time series of at least one of the signal estimators by at least one of:
Defining a plurality of sub-series for at least one of the time series or for the extended time series and, for each sub-series, determine statistical properties,
Fitting one or more parametrized curves comprising one or more parameters to at least one of the time series or to the extended time series, and
Defining a plurality of sub-series for at least one of the time series or for the extended time series and, for each sub-series, fit one or more parametrized curves comprising one or more parameters in order to obtain fitted parameters.

8. The method according to claim 1, wherein evaluating the detected time-dependent signal with the evaluation device comprises detecting the time-dependent signal during a time t, and wherein analysing the time-dependency of the signal comprises the steps of:
Dividing the signal of time t into time intervals of length T,
For each time interval j=1, . . . t/T, performing the steps of:
calculating the digital integral of the signal within each time interval;
divide the time interval into at least one number of subintervals;
for each subinterval, detrend the digital integral of the signal by applying a detrending algorithm to the digital integral of the signal;
for the at least one number of subintervals, combine the subintervals into a detrended interval; and
determine at least one generalized mean of the detrended signal of the detrended interval for the at least one number of subintervals, whereby at least one signal parameter is obtained.

9. The method according to claim 8, wherein at least one signal parameter is derived from at least one of ratios or differences between at least two signal parameters.

10. The method according to claim 1, further comprising executing a feature selection algorithm to automatically select a subset of signal parameters so as to obtain the input vector being fed to the linking algorithm.

11. The method according to claim 1, wherein a drift cancellation algorithm is applied to the signal prior to the derivation of the signal parameters from the signal.

12. The method according to claim 1, wherein the linking algorithm is a regression algorithm or a classification algorithm.

13. The method according to claim 1, wherein the linking algorithm is a machine learning algorithm.

14. The method according to claim 1, wherein the particle is at least one of a cell such as a prokaryotic cell or an eukaryotic cell, a spore, a virus, a phage, a vesicle, a peptide, a protein, a polysaccharide, a lipid, a glucide, a nucleic acid or a co-polymer thereof, a protein-RNA co-polymer, a RNA-DNA co-polymer, a protein-DNA co-polymers, a RNA/DNA-protein co-polymer, a protein-protein co-polymer, a capsule, an organelle, or a nanodevice.

15. The method according to claim 1, wherein the activity indicator is indicative of at least one of a metabolic activity of the particle, a physiological state of the particle, a respiration level of the particle, an ATP level of the particle, a ratio of NADH/NAD+ of the particle, a membrane potential of the particle, a growth rate of the particle, a kill rate of the particle, an interaction of the particle with other particles, and an interaction of the particle with an environmental factor of the particle or a combination of any of those.

16. The method according to claim 1, wherein the particle is subjected to at least one of: at least one chemical stimulus and/or at least one physical stimulus.

17. The method according to claim 16, wherein the activity indicator is determined at least one of before, during or after the particle is subjected to at least one of the chemical stimulus or the physical stimulus, and wherein the thus determined activity indicators are compared with one another.

18. A method of generating a training data set of a machine learning algorithm comprising:
a) Bringing at least one particle into contact with a flexible support,
b) Detecting at least one time-dependent signal that is indicative of deflections of the flexible support due to motions of the at least one particle, using a detection device, and
c) Evaluating the detected time-dependent signal with an evaluation device, wherein the evaluation of the detected time-dependent signal comprises analysing a time-dependency of the signal in order to derive a plurality of signal parameters from the signal that characterise a variation of the signal as a function of time,
d) Performing an independent measurement in order to derive at least one associated activity indicator that is indicative of a motional activity of the particle,
e) Saving the plurality of signal parameters and the at least one associated activity indicator in the training data set, and
f) Repeating steps a) to e) for a plurality of particles exhibiting different motional activities.

19. A method of training a machine learning algorithm, wherein the machine learning algorithm is trained with a training data set generated by the method of claim 18.

20. The method according to claim 4, wherein the geometrical property is an extreme point of the power spectral density function, an inflection point of the power spectral density function, an area under a curve described by the power spectral density function or at least one of a horizontal or a vertical distance between them.

21. The method according to claim 5, wherein, for all periodograms, at least one percentile is determined for at least one frequency range, whereby the signal estimator is obtained.

22. The method of claim 6, wherein at least one of:
at least one distance between the values of the empirical probability density function and values of a theoretical probability density function is determined; or
at least one of: at least one geometrical property or various moments of the empirical probability density function is determined.

23. The method according to claim 7, wherein at least one of:
for each sub-series, determine furthermore at least one of ratios, differences and ratios of differences of the statistical properties, or
for each sub-series, furthermore determine ratios, differences and ratios of differences of the fitted parameters.

24. The method according to claim 10, wherein the feature selection algorithm is configured to select the subset of signal parameters in such a manner that the subset is optimal to a multi-objective criterion.

25. The method according to claim 11, wherein at least one of:
the drift cancellation algorithm comprises a curve-fitting procedure that models a drift component of the signal or a high-pass filtering, or
the drift cancellation algorithm is a detrending algorithm.

26. The method according to claim 16, wherein at least one of:
the particle is a cell and wherein the chemical stimulus is an addition of a drug such as an antibiotic or a compound affecting the metabolism or viability of the cell or a change in an environmental condition such as a culture condition, or
the physical stimulus is an application of stress.
